(19) United States
(12) Patent Application Publication
(10) Pub. No.: US 2013/0280214 A1
(43) Pub. Date: Oct. 24, 2013

(54) POLYCYCLIC HETEROCYCLE DERIVATIVES AND METHODS OF USE THEREOF FOR THE TREATMENT OF VIRAL DISEASES

(71) Applicants: Joseph P. Vacca, Telford, PA (US); Craig A. Coburn, Royersford, PA (US); David B. Olsen, Royersford, PA (US); Joseph A. Kozlowski, Princeton, NJ (US); Stuart B. Rosenblum, West Orange, NJ (US)

(72) Inventors: Joseph P. Vacca, Telford, PA (US); Craig A. Coburn, Royersford, PA (US); David B. Olsen, Royersford, PA (US); Joseph A. Kozlowski, Princeton, NJ (US); Stuart B. Rosenblum, West Orange, NJ (US)

(73) Assignee: Merck Sharp & Dohme Corp., Rahway, NJ (US)

(21) Appl. No.: 13/876,908

(22) PCT Filed: Sep. 28, 2011
(86) PCT No.: PCT/US11/53562

§ 371 Date: Jun. 26, 2013

Related U.S. Application Data
(60) Provisional application No. 61/387,825, filed on Sep. 29, 2010.

(51) Int. Cl. A61K 38/07; A61K 31/7056; A61K 38/21; A61K 38/05; A61K 45/06

(52) U.S. Cl. CPC A61K 38/07; A61K 38/05; A61K 45/06; A61K 38/212; A61K 31/7056
USPC 424/85.7; 514/4.3; 424/85.4

(57) ABSTRACT

The present invention relates to Polycyclic Heterocycle Derivatives, such as compound 1: (1) compositions comprising the Polycyclic Heterocycle Derivatives, and methods of using the Polycyclic Heterocycle Derivatives for treating or preventing HCV infection in a patient.

### FIELD OF THE INVENTION

The present invention relates to Polycyclic Heterocycle Derivatives, compositions comprising the Polycyclic Heterocycle Derivatives, and methods of using the Polycyclic Heterocycle Derivatives for treating or preventing HCV infection in a patient.

### BACKGROUND OF THE INVENTION

Hepatitis C virus (HCV) is a major human pathogen. A substantial fraction of these HCV-infected individuals develop serious progressive liver disease, including cirrhosis and hepatocellular carcinoma, which are often fatal. HCV is a (+)-sense single-stranded enveloped RNA virus that has been implicated as the major causative agent in non-A, non-B hepatitis (NANBH), particularly in blood-associated NANBH (BB-NANBH) (see, International Publication No. WO 89/04669 and European Patent Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

It is well-established that persistent infection of HCV is related to chronic hepatitis, and as such, inhibition of HCV replication is a viable strategy for the prevention of hepatocellular carcinoma. Current therapies for HCV infection include α-interferon monotherapy and combination therapy comprising α-interferon and ribavirin. These therapies have been shown to be effective in some patients with chronic HCV infection, but suffer from poor efficacy and unfavorable side-effects and there are currently efforts directed to the discovery of HCV replication inhibitors that are useful for the treatment and prevention of HCV related disorders.

Current research efforts directed toward the treatment of HCV includes the use of antisense oligonucleotides, free bile acids (such as ursodeoxycholic acid and chenodeoxycholic acid) and conjugated bile acids (such as tauroursodeoxycholic acid). Phosphonoformic acid esters have also been proposed as potentially useful for the treatment of various viral infections, including HCV. Vaccine development, however, has been hampered by the high degree of viral strain heterogeneity and immune evasion and the lack of protection against reinfection, even with the same inoculum.

In light of these treatment hurdles, the development of small-molecule inhibitors directed against specific viral targets has become a major focus of anti-HCV research. The determination of crystal structures for NS3 protease, NS3 RNA helicase, NS5A, and NS5B polymerase, with and without bound ligands, has provided important structural insights useful for the rational design of specific inhibitors.

Recent attention has been focused toward the identification of inhibitors of HCV NS5A. HCV NS5A is a 447 amino acid phosphoprotein which lacks a defined enzymatic function. It runs as 56 kd and 58 kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. See U.S. Patent Publication Nos. US20080311075, US20080044379, US20080050336, US20080044380, US20090202483 and US2009020478, and International Patent Publication Nos. WO 10/065681, WO 10/065668, and WO 10/065674.

Other HCV NS5A inhibitors and their use for reducing viral load in HCV infected humans have been described in U.S. Patent Publication No. US20060276511.

### SUMMARY OF THE INVENTION

The present invention provides compositions comprising Compounds 1-14 of Table 1 (the “Polycyclic Heterocycle Derivatives”) and methods of using the Polycyclic Heterocycle Derivatives for inhibiting HCV NS5A activity or for preventing and/or treating infection by HCV in a patient in need thereof.

TABLE 1

| No. | Structure |
|---|---|
| 1 | 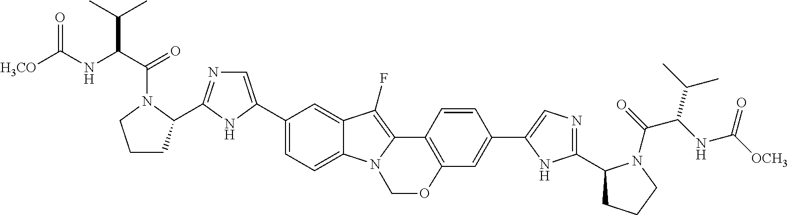 [SMILES: COC(=O)N[C@H](C(=O)N1CCC[C@H]1c1ncc(-c2ccc3c(c2)OCn2c-3c(F)c3cc(-c4cnc([C@@H]5CCCN5C(=O)[C@@H](NC(=O)OC)C(C)C)[nH]4)ccc32)[nH]1)C(C)C] |
| 2 | 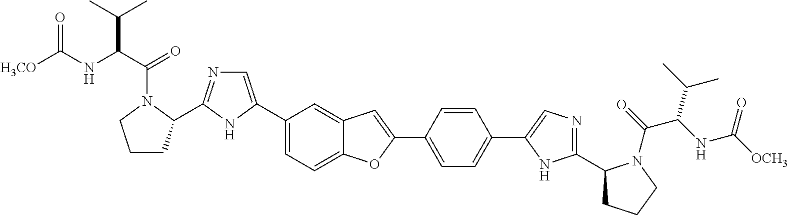 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C(C)C)[nH]5)ccc4o3)cc2)[nH]1)C(C)C] |
| 3 | 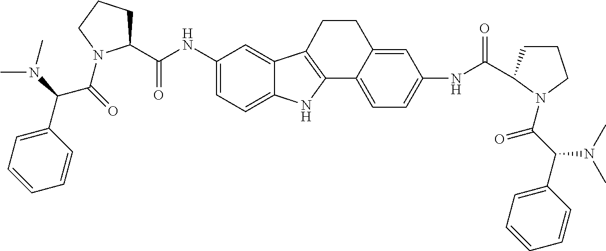 |
| 4 | 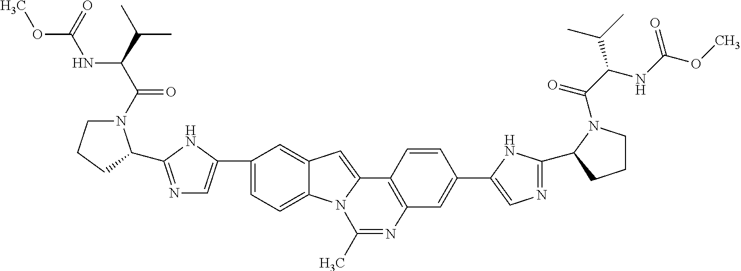 |
| 5 | 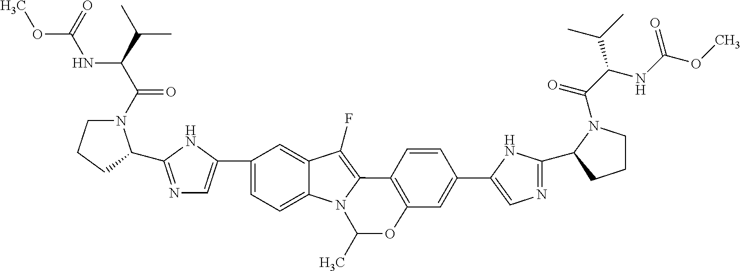 |
| 6 | 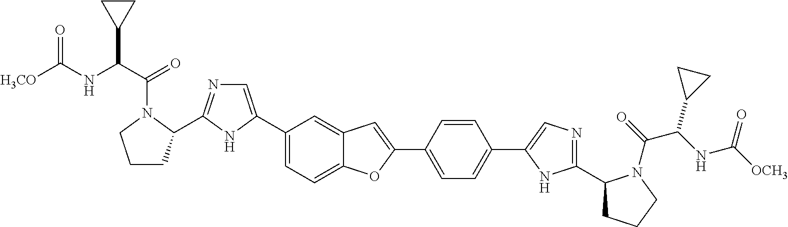 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C6CC6)[nH]5)ccc4o3)cc2)[nH]1)C1CC1] |
| 7 | 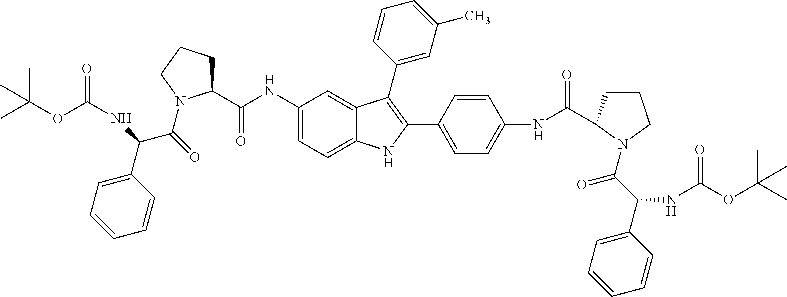 [SMILES: Cc1cccc(-c2c(-c3ccc(NC(=O)[C@@H]4CCCN4C(=O)[C@H](NC(=O)OC(C)(C)C)c4ccccc4)cc3)[nH]c3ccc(NC(=O)[C@@H]4CCCN4C(=O)[C@H](NC(=O)OC(C)(C)C)c4ccccc4)cc23)c1] |
| 8 | 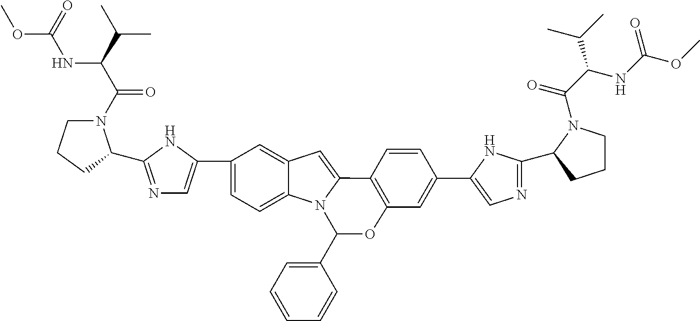 |
| 9 | 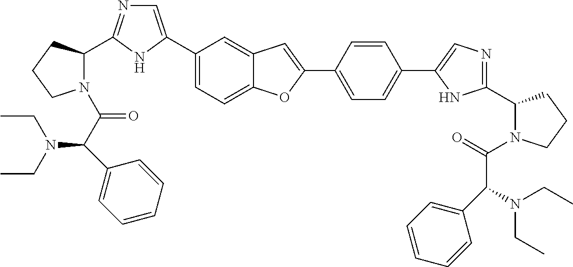 [SMILES: CCN(CC)[C@@H](C(=O)N1CCC[C@H]1c1ncc(-c2ccc(-c3cc4cc(-c5cnc([C@@H]6CCCN6C(=O)[C@@H](c6ccccc6)N(CC)CC)[nH]5)ccc4o3)cc2)[nH]1)c1ccccc1] |
| 10 | 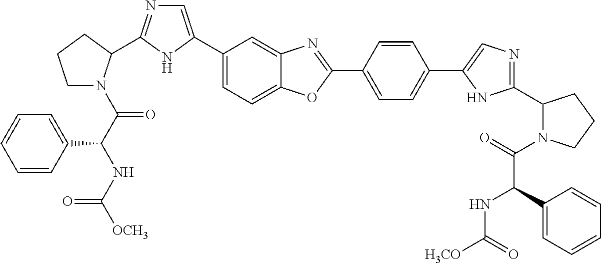 [SMILES: COC(=O)N[C@@H](C(=O)N1CCCC1c1ncc(-c2ccc(-c3nc4cc(-c5cnc(C6CCCN6C(=O)[C@H](NC(=O)OC)c6ccccc6)[nH]5)ccc4o3)cc2)[nH]1)c1ccccc1] |
| 11 | 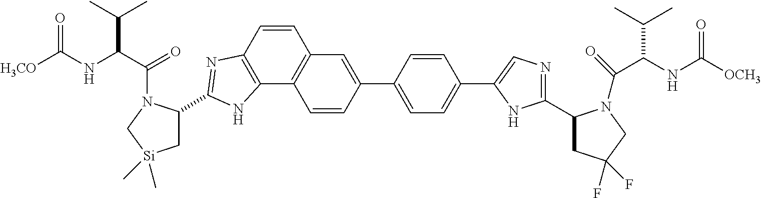 |
| 12 | 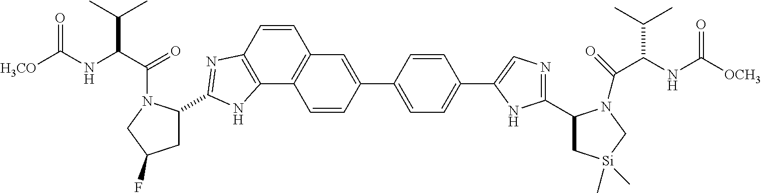 |
| 13 | 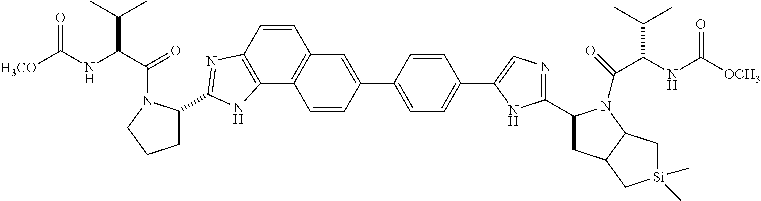 |
| 14 | 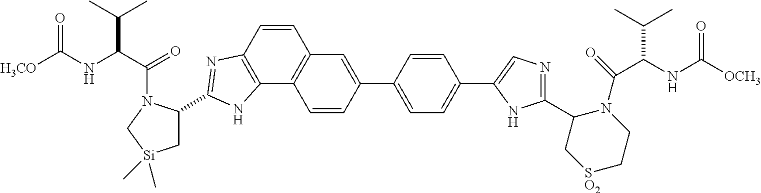 |

and pharmaceutically acceptable salts thereof.

The Compounds of Table 1 and pharmaceutically acceptable salts thereof can be useful, for example, for inhibiting HCV viral replication or replicon activity, and for treating or preventing HCV infection in a patient. Without being bound by any specific theory, it is believed that the Compounds of Table 1 inhibit HCV viral replication by inhibiting HCV NS5A.

Accordingly, the present invention provides pharmaceutical compositions comprising: (1) a pharmaceutically acceptable carrier; (ii) a compound selected from Table 1, or a pharmaceutically acceptable salt thereof, and (iii) a first additional therapeutic agent, selected from compounds F1-F28:

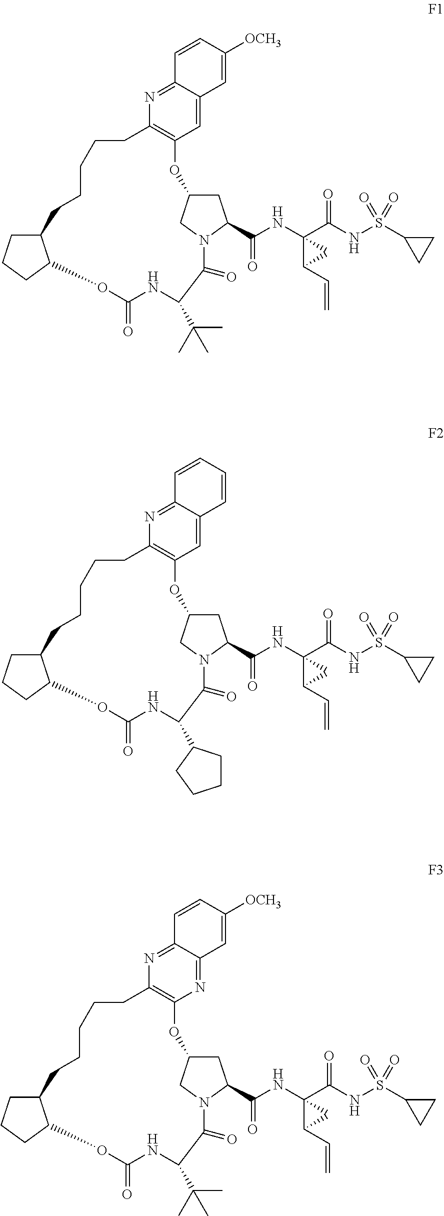

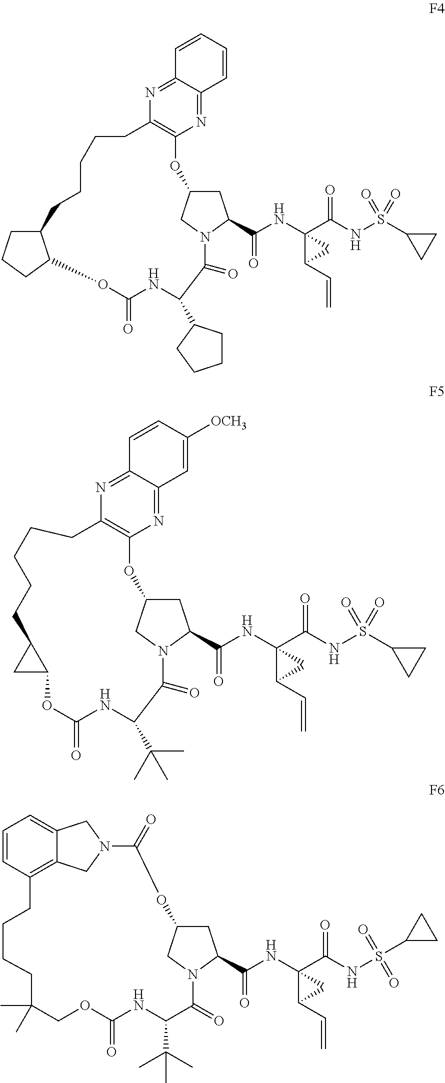

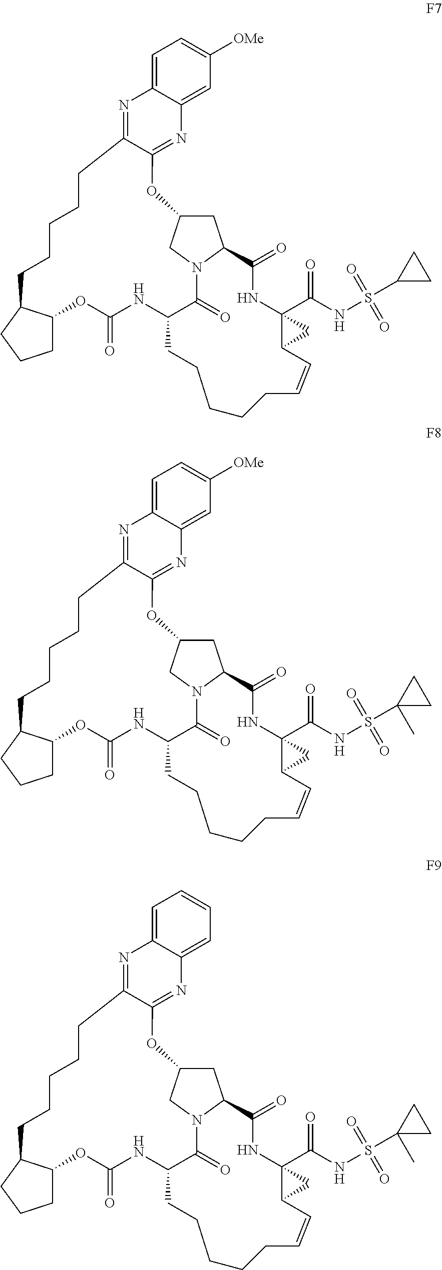

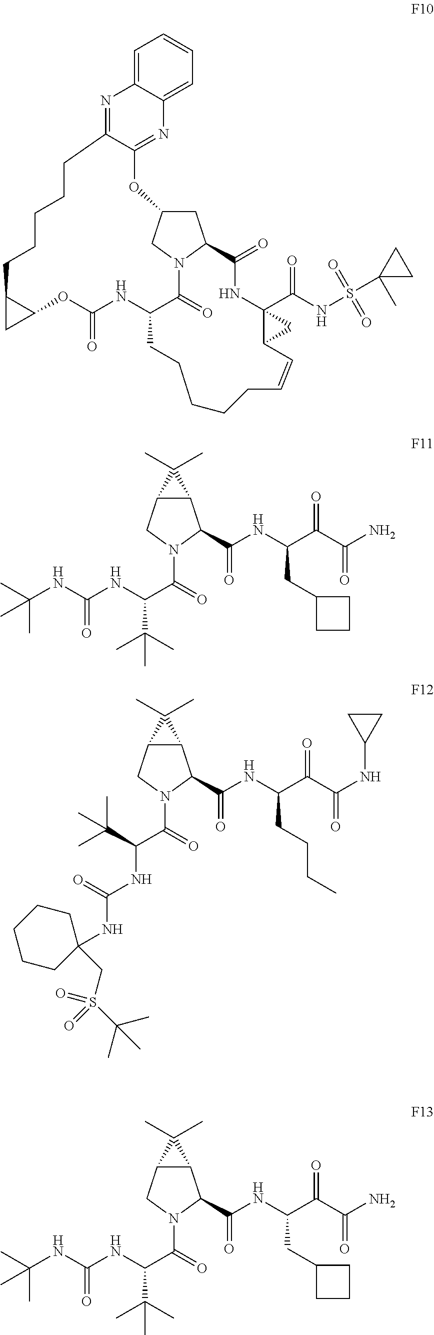

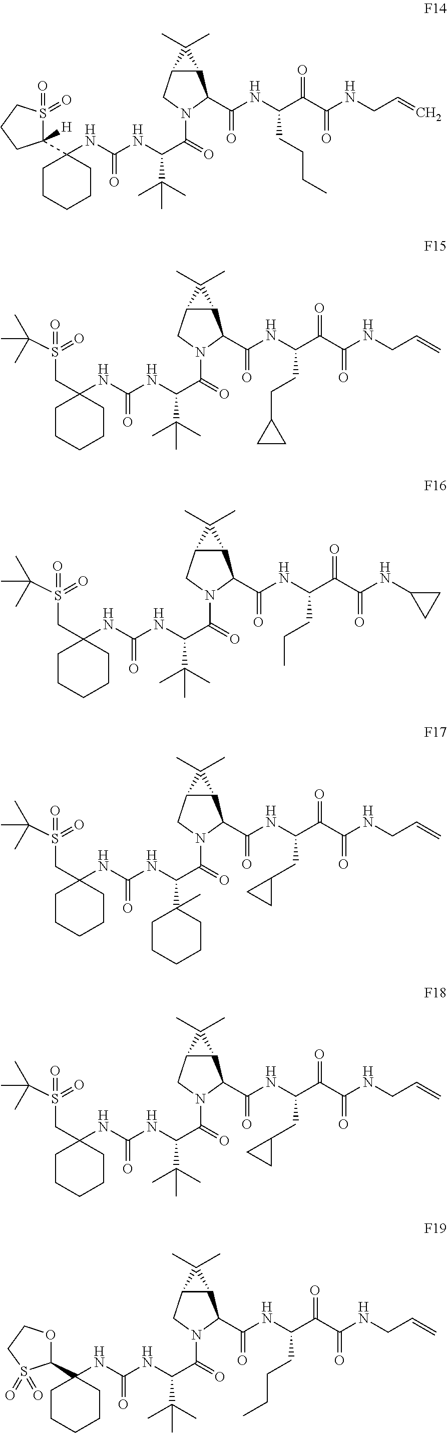

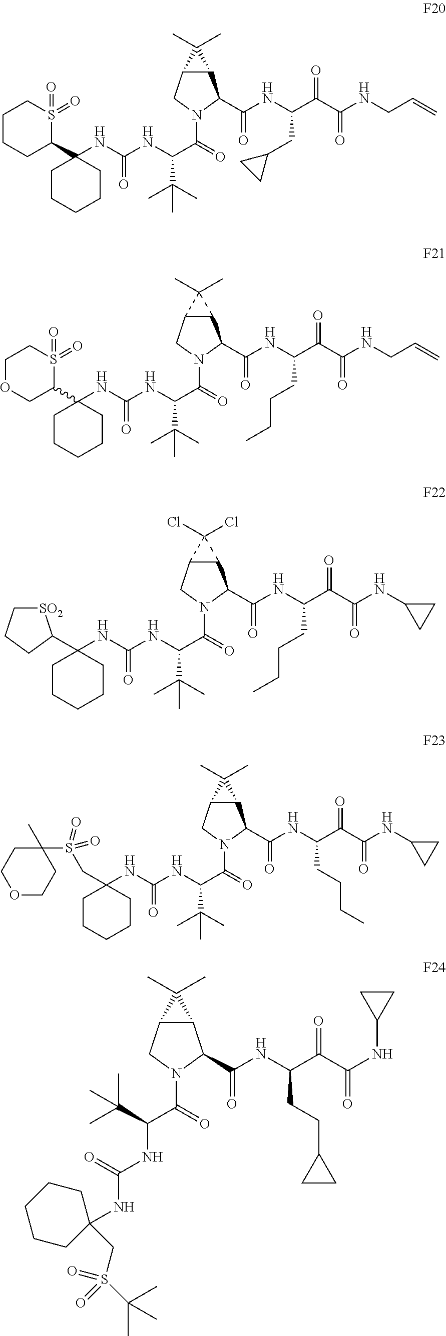

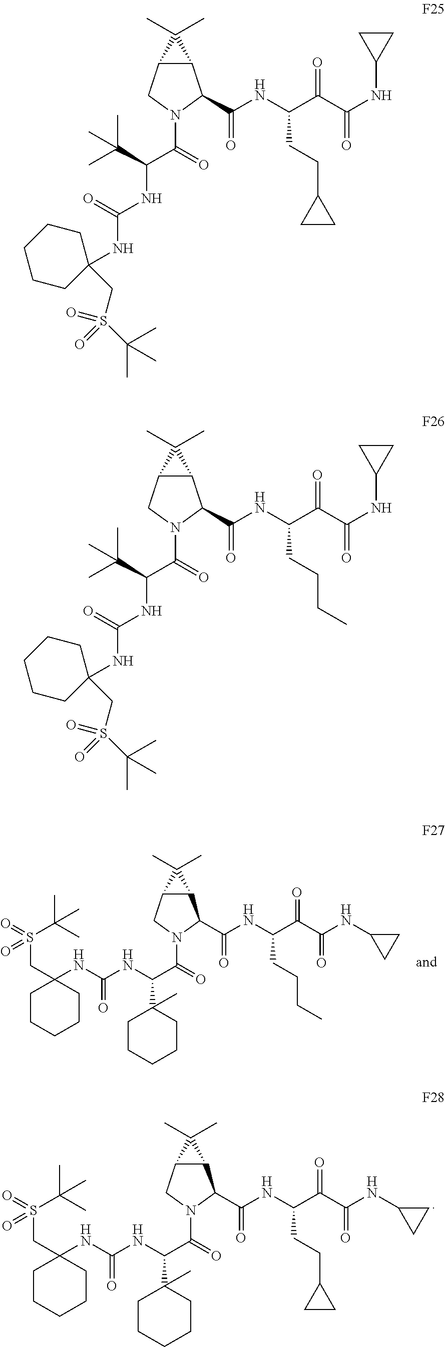 [SMILES: CC(C)(C)[C@H](NC(=O)NC1(CS(=O)(=O)C(C)(C)C)CCCCC1)C(=O)N1C[C@H]2[C@@H]([C@H]1C(=O)N[C@@H](CCC1CC1)C(=O)C(=O)NC1CC1)C2(C)C ; CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1 ; CCCC[C@H](NC(=O)[C@@H]1[C@@H]2[C@H](CN1C(=O)[C@@H](NC(=O)NC1(CS(=O)(=O)C(C)(C)C)CCCCC1)C1(C)CCCCC1)C2(C)C)C(=O)C(=O)NC1CC1 ; CC1([C@H](NC(=O)NC2(CS(=O)(=O)C(C)(C)C)CCCCC2)C(=O)N2C[C@H]3[C@@H]([C@H]2C(=O)N[C@@H](CCC2CC2)C(=O)C(=O)NC2CC2)C3(C)C)CCCCC1]

or a pharmaceutically acceptable salt thereof, wherein the amounts of the compound of Table 1 and the first additional therapeutic agent are together effective to treat HCV infection in a patient.

The present invention also provides methods for treating or preventing HCV, the method comprising administering to the patient: (i) a compound selected from Table 1 or a pharmaceutically acceptable salt thereof, and (ii) a first additional therapeutic agent that is selected from compounds F1-F28 or a pharmaceutically acceptable salt thereof, wherein the amounts administered of the compound of Table 1 and the first additional therapeutic agent are together effective to treat the HCV infection.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### BRIEF DESCRIPTION OF THE DRAWINGS

FIG. 1 illustrates the addition of a first test compound in the 384 well low dead volume plate according to the RHEPLUC assay protocol of Example 2. The designation “M” in row P of the plate indicates that these wells are to contain medium only and no cells. The arrows indicate the direction of the dilution of the first test compound, where high indicates the highest concentration tested and low indicates the lowest concentration tested.

FIG. 2 illustrates the addition of a second test compound in the 384 well low dead volume plate according to the RHEPLUC assay protocol of Example 2. The designation “M” represents wells that contain only complete growth media and no cells. The arrows indicate the direction of the dilution of the second test compound, where high indicates the highest concentration tested and low indicates the lowest concentration tested.

FIG. 3 illustrates the addition of the first and second test compounds in the 384 well “200× test compound mix plate” according to the Combination Study protocol of Example 2. The designation “1+2” represents wells containing a mixture of the first and second test compounds; “1” represents wells containing first test compound only; “2” represents wells containing second test compound only; “C” represents wells that contain only Compound A (100% inhibition control); “D” represents wells containing DMSO only (0% inhibition control); and “M” represents wells that contain only complete growth media and no cells.

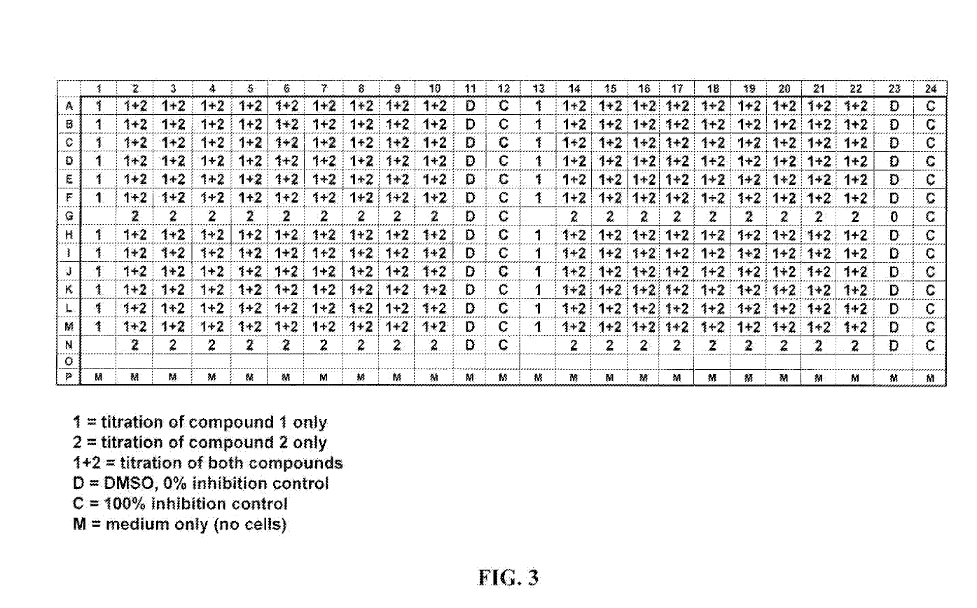

FIG. 4 illustrates the combination effects of Compound 2 and Compound F5 on genotype 1a replicon cells according to the protocol of Example X. The X-axis represents concentration of Compound 2 (log nM) and the Y-axis represents cycle of threshold. Graphically, * represents Compound F5 at 0 nM; Δ represents Compound F5 at 0.019 nM; ∘ represents Compound F5 at 0.156 nM; ▾ represents Compound F5 at 0.625 nM; ▴ represents Compound F5 at 1.25 nM; and represents Compound F5 at 5 nM.

FIG. 5 illustrates the long-term combination effects of Compound 2 and Compound F5, alone and in combination, on genotype 1a replicon cells. The x-axis represents time in weeks and the y-axis represents the log decrease in HCV RNA. Graphically, ▾ represents DMSO, ▪ represents Compound 2 (1×EC₉₀), ▴ represents Compound F5 (3×EC₉₀) and represents the combination of Compound 2 (1×EC₉₀) and Compound F5 (3×EC₉₀). The gray shaded area represents the level of detection for the method used (approximately −3.5 log).

FIG. 6 illustrates the effects of Compound 2 and Compound F5, alone and in combination, on emergence of resistance in genotype 1a replicon cells. The x-axis represents the concentration of Compound 2 at 0, 1, 10, 100 and 1000 multiples of the EC₉₀ of this compound (as determined using the method described in Example 5). The y-axis represents the concentration of Compound F5 at 0, 1, 10 and 100 multiples of the EC₉₀ of this compound (as determined using the method described in Example 5). The data represents the approximate number of surviving cell colonies after treatment with Compound 2 and/or Compound F5.

### DETAILED DESCRIPTION OF THE INVENTION

The present invention relates to Polycyclic Heterocycle Derivatives, compositions comprising at least one Polycyclic Heterocycle Derivative, and methods of using the Polycyclic Heterocycle Derivatives for treating or preventing HCV infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated.

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Polycyclic Heterocycle Derivative and one or more additional therapeutic agents, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Polycyclic Heterocycle Derivative or a pharmaceutically acceptable salt or solvate of the compound. The transformation may occur by various mechanisms (e.g., by metabolic or chemical processes), such as, for example, through hydrolysis in blood.

If a Polycyclic Heterocycle Derivative incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇)cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl; carboxy(C₁-C₆)alkyl; amino(C₁-C₄)alkyl or mono-N— or di-N,N—(C₁-C₆)alkylaminoalkyl; —C(Y⁴)Y^{5′} wherein Y⁴ is H or methyl and Y⁵ is mono-N- or di-N,N—(C₁-C₆)alkylamino morpholine; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C ₁₋₂₀ alcohol or reactive derivative thereof, or by a 2,3-di(C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Polycyclic Heterocycle Derivatives can form salts which are also within the scope of this invention. Reference to a Polycyclic Heterocycle Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Polycyclic Heterocycle Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Table 1 may be formed, for example, by reacting a Polycyclic Heterocycle Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, dihydrochlorides, hydrobromides, hydroiodides, lactates, maleates, methanesulfonates (“mesylates”), dimesylates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

In one embodiment, the Polycyclic Heterocycle Derivatives are in the form of a dihydrochloride salt. In another embodiment, the Polycyclic Heterocycle Derivatives are in the form of a dimesylate salt.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Polycyclic Heterocycle Derivatives may be atropisomers (e.g., substituted biaryls) and are considered as part of this invention. Enantiomers can also be directly separated using chiral chromatographic techniques.

It is also possible that the Polycyclic Heterocycle Derivatives may exist in different tautomeric forms, and all such fowls are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Polycyclic Heterocycle Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Table 1, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Table 1. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Table 1 can be prepared without undue experimentation by conventional techniques well known to those skilled in the art or by processes analogous to those described in the Schemes and Examples herein using appropriate isotopically-enriched reagents and/or intermediates. In one embodiment, a Compound of Table 1 has one or more of its hydrogen atoms replaced with deuterium.

Polymorphic forms of the Polycyclic Heterocycle Derivatives, and of the salts, solvates, hydrates, esters and prodrugs of the Polycyclic Heterocycle Derivatives, are intended to be included in the present invention.

The following abbreviations are used below and have the following meanings: Dulbecco's PBS is Dulbecco's phosphate-buffered saline; DMEM is Dulbecco's Modified Eagle Medium; DMSO is dimethylsulfoxide; G418 is (2R,3S,4R,5R,6S)-5-amino-6-[(1R,2S,3S,4R,6S)-4,6-diamino-3-[(2R,3R,4R,5R)-3,5-dihydroxy-5-methyl-4-methylaminooxan-2-yl]oxy-2-hydroxycyclohexyl]oxy-2-(1-hydroxyethyl)oxane-3,4-diol; and PBS is phosphate-buffered saline.

### The Compounds of Table (I)

The present invention provides Polycyclic Heterocycle Derivatives of Table 1:

TABLE 1

| No. | Structure | MS |
|---|---|---|
| 1 | 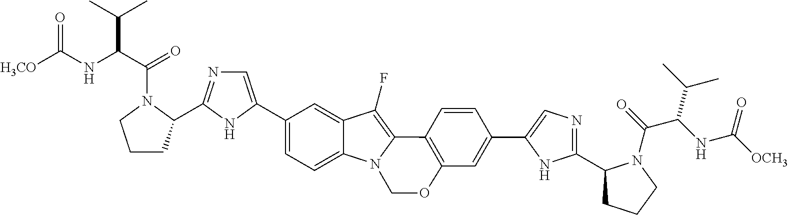 [SMILES: COC(=O)N[C@H](C(=O)N1CCC[C@H]1c1ncc(-c2ccc3c(c2)OCn2c-3c(F)c3cc(-c4cnc([C@@H]5CCCN5C(=O)[C@@H](NC(=O)OC)C(C)C)[nH]4)ccc32)[nH]1)C(C)C] | 825 |
| 2 | 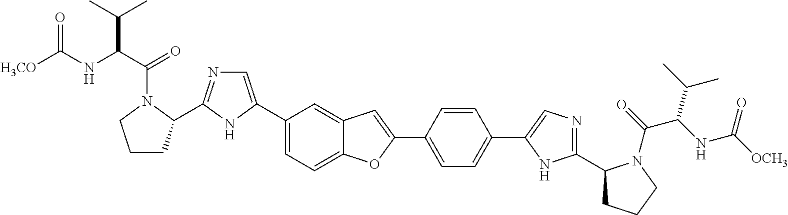 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C(C)C)[nH]5)ccc4o3)cc2)[nH]1)C(C)C] | 780 |
| 3 | 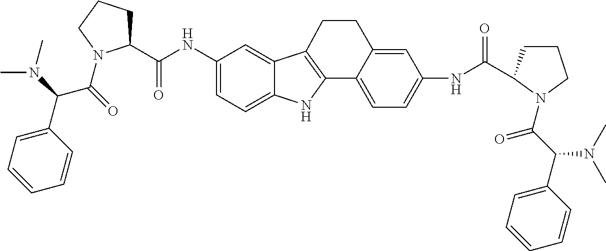 | 765 |
| 4 | 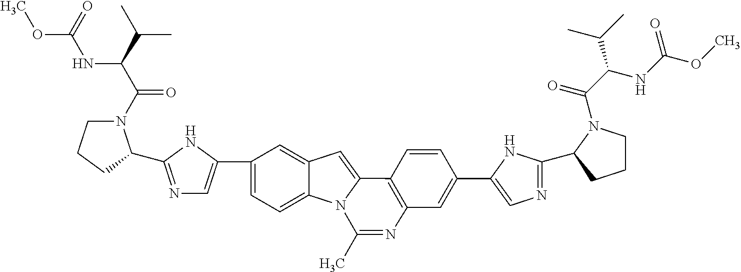 | 818 |
| 5 | 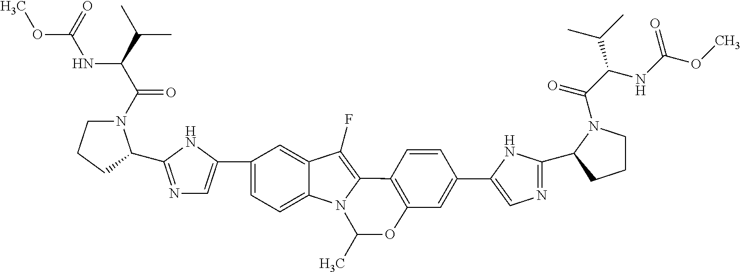 | 839 |
| 6 | 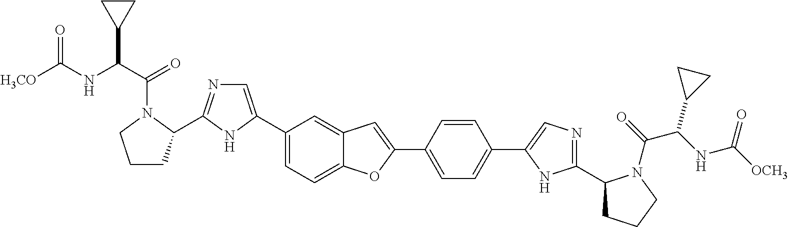 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C6CC6)[nH]5)ccc4o3)cc2)[nH]1)C1CC1] | 775 |
| 7 | 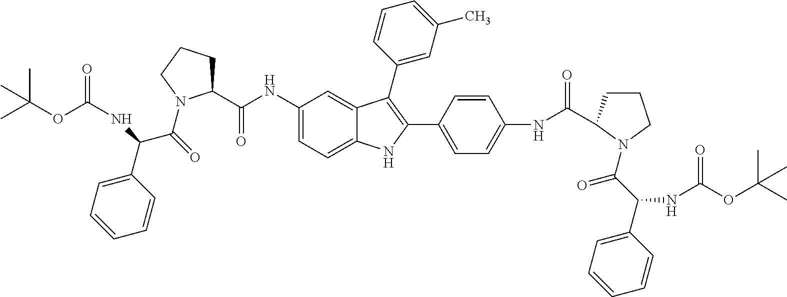 [SMILES: Cc1cccc(-c2c(-c3ccc(NC(=O)[C@@H]4CCCN4C(=O)[C@H](NC(=O)OC(C)(C)C)c4ccccc4)cc3)[nH]c3ccc(NC(=O)[C@@H]4CCCN4C(=O)[C@H](NC(=O)OC(C)(C)C)c4ccccc4)cc23)c1] | 974 |
| 8 | 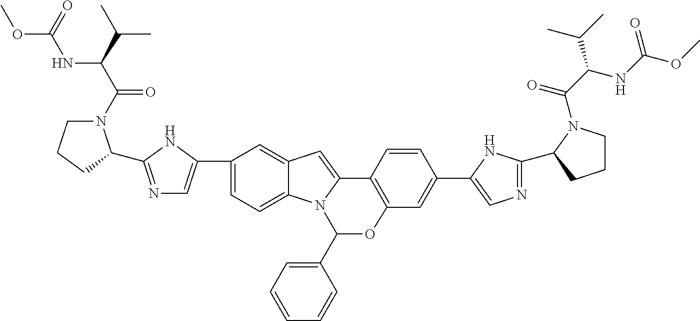 | 883 |
| 9 | 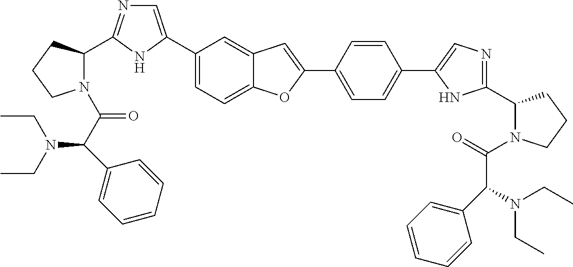 [SMILES: CCN(CC)[C@@H](C(=O)N1CCC[C@H]1c1ncc(-c2ccc(-c3cc4cc(-c5cnc([C@@H]6CCCN6C(=O)[C@@H](c6ccccc6)N(CC)CC)[nH]5)ccc4o3)cc2)[nH]1)c1ccccc1] | 844 |
| 10 | 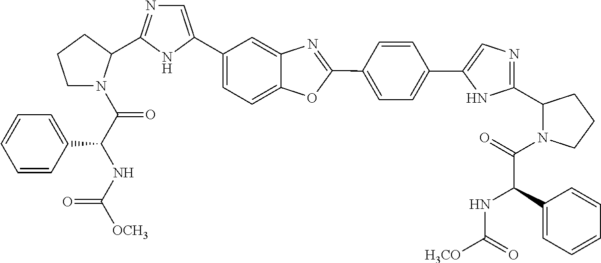 [SMILES: COC(=O)N[C@@H](C(=O)N1CCCC1c1ncc(-c2ccc(-c3nc4cc(-c5cnc(C6CCCN6C(=O)[C@H](NC(=O)OC)c6ccccc6)[nH]5)ccc4o3)cc2)[nH]1)c1ccccc1] | 780 |
| 11 | 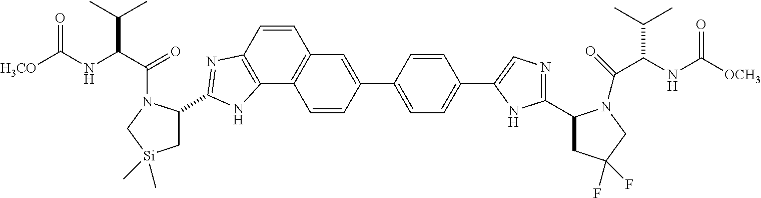 | 842 |
| 12 | 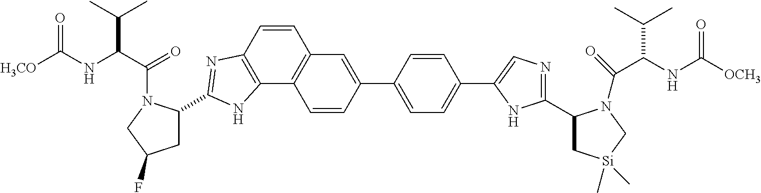 | 824 |
| 13 | 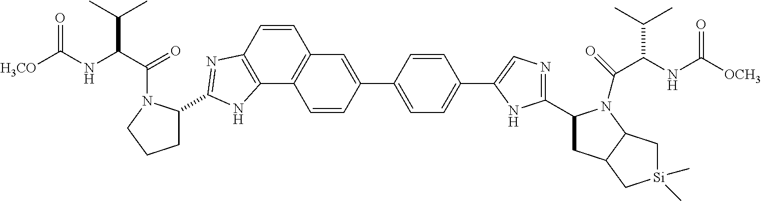 | 846 |
| 14 | 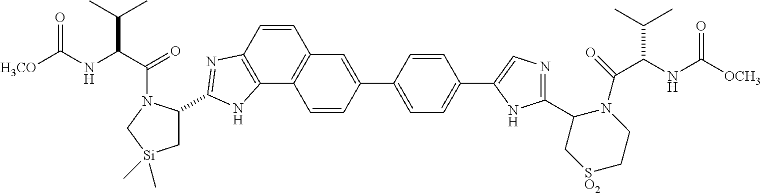 | 870 |

and pharmaceutically acceptable salts thereof.

In one embodiment, the Polycyclic Heterocycle Derivatives are in substantially purified form.

In another embodiment, the present invention includes a pharmaceutical composition of the present invention for use in (i) inhibiting HCV replication or (ii) treating HCV infection and/or reducing the likelihood or severity of symptoms of HCV infection. In these uses, the compounds of the present invention can optionally be employed in combination with one or more additional therapeutic agents, selected from HCV antiviral agents, anti-infective agents, and immunomodulators.

In another embodiment, the present invention also includes a pharmaceutical composition of the present invention for use (i) in, (ii) as a medicament for, or (iii) in the preparation of a medicament for: (a) medicine, (b) inhibiting HCV replication or (c) treating HCV infection and/or reducing the likelihood or severity of symptoms of HCV infection. In these uses, the compounds of the present invention can optionally be employed in combination with one or more additional therapeutic agents, selected from HCV antiviral agents, anti-infective agents, and immunomodulators.

### Uses of the Polycyclic Heterocycle Derivatives

The Polycyclic Heterocycle Derivatives are useful in human and veterinary medicine for treating or preventing a viral infection in a patient. In one embodiment, the Polycyclic Heterocycle Derivatives can be inhibitors of viral replication. In another embodiment, the Polycyclic Heterocycle Derivatives can be inhibitors of HCV replication. Accordingly, the Polycyclic Heterocycle Derivatives are useful for treating viral infections, such as HCV. In accordance with the invention, the Polycyclic Heterocycle Derivatives can be administered to a patient in need of treatment or prevention of a viral infection.

Accordingly, in one embodiment, the invention provides methods for treating a viral infection in a patient comprising administering to the patient an effective amount of at least one Polycyclic Heterocycle Derivative or a pharmaceutically acceptable salt thereof and one or more additional therapeutic agents that are not Compounds of Table 1.

### Treatment or Prevention of a Flaviviridae Virus

The Polycyclic Heterocycle Derivatives can be useful in combination with one or more additional therapeutic agents for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-borne encephalitis, West Nile encephalitis, yellow fever and Hepatitis C Virus (HCV) infection.

In one embodiment, the Flaviviridae infection being treated is hepatitis C virus infection.

### Treatment or Prevention of HCV Infection

The Polycyclic Heterocycle Derivatives can be useful in combination with one or more additional therapeutic agents for the inhibition of HCV (e.g., HCV NS5A), the treatment of HCV infection and/or reduction of the likelihood or severity of symptoms of HCV infection and the inhibition of HCV viral replication and/or HCV viral production in a cell-based system. For example, the Polycyclic Heterocycle Derivatives are useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of (i) a Polycyclic Heterocycle Derivative or a pharmaceutically acceptable salt thereof and (ii) a first additional therapeutic agent as defined below or a pharmaceutically acceptable salt thereof. In a specific embodiment, the amounts administered of the Polycyclic Heterocycle Derivative and the first additional therapeutic agent are together effective to treat or prevent infection by HCV in the patient. In another specific embodiment, the amounts administered of the Polycyclic Heterocycle Derivative and the first additional therapeutic agent are together effective to inhibit HCV viral replication and/or viral production in the patient. In another embodiment, the amounts administered of the Polycyclic Heterocycle Derivative and the first additional therapeutic agent are those that render each of the Polycyclic Heterocycle Derivative and the first additional therapeutic agent alone effective.

In another embodiment, the invention provides methods for treating HCV infection in a patient, the methods comprising administering to the patient an effective amount of (i) a Polycyclic Heterocycle Derivative or a pharmaceutically acceptable salt thereof; (ii) a first additional therapeutic agent as defined below or a pharmaceutically acceptable salt thereof, and (iii) a second additional therapeutic agent as defined below or a pharmaceutically acceptable salt thereof. In a specific embodiment, the amounts administered of the Polycyclic Heterocycle Derivative, the first additional therapeutic agent and the second additional therapeutic agent are together effective to treat or prevent infection by HCV in the patient. In another specific embodiment, the amounts administered of the Polycyclic Heterocycle Derivative, the first additional therapeutic agent and the second additional therapeutic agent are together effective to inhibit HCV viral replication and/or viral production in the patient.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### The Additional Therapeutic Agents

In one embodiment, the present invention provides methods for treating a viral infection in a patient, the method comprising administering to the patient: (i) at least one Polycyclic Heterocycle Derivative or a pharmaceutically acceptable salt thereof, and (ii) a first additional therapeutic agent selected from compounds F1-F28, or a pharmaceutically acceptable salt thereof, wherein the amounts administered are together effective to treat or prevent a viral infection.

In another embodiment, the present invention provides methods for treating a viral infection in a patient, the method comprising administering to the patient: (i) at least one Polycyclic Heterocycle Derivative or a pharmaceutically acceptable salt thereof, (ii) a first additional therapeutic agent selected from compounds F1-F28, or a pharmaceutically acceptable salt thereof; and (iii) a second additional therapeutic agent, defined below herein, or a pharmaceutically acceptable salt thereof, wherein the amounts administered are together effective to treat or prevent a viral infection.

When administering a combination therapy of the invention to a patient, the active agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Polycyclic Heterocycle Derivative and a first additional therapeutic agent may be present in fixed amounts (dosage amounts) in a single dosage unit (e.g., a capsule, a tablet and the like).

In one embodiment, the Polycyclic Heterocycle Derivative is administered during a time when the additional therapeutic agent(s) exert their prophylactic or therapeutic effect, or vice versa.

In another embodiment, the Polycyclic Heterocycle Derivative and the additional therapeutic agent(s) are administered in doses commonly employed when such agents are used as monotherapy for treating a viral infection.

In another embodiment, the Polycyclic Heterocycle Derivative and the additional therapeutic agent(s) are administered in doses lower than the doses commonly employed when such agents are used as monotherapy for treating a viral infection.

In still another embodiment, the Polycyclic Heterocycle Derivative and the additional therapeutic agent(s) act synergistically and are administered in doses lower than the doses commonly employed when such agents are used as monotherapy for treating a viral infection.

In one embodiment, the Polycyclic Heterocycle Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Polycyclic Heterocycle Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Polycyclic Heterocycle Derivative and the additional therapeutic agent(s) may inhibit the resistance of a viral infection to these agents.

### First Additional Therapeutic Agents

First additional therapeutic agents useful in the present compositions and methods include compounds F1-F28, depicted immediately below, and pharmaceutically acceptable salts thereof.

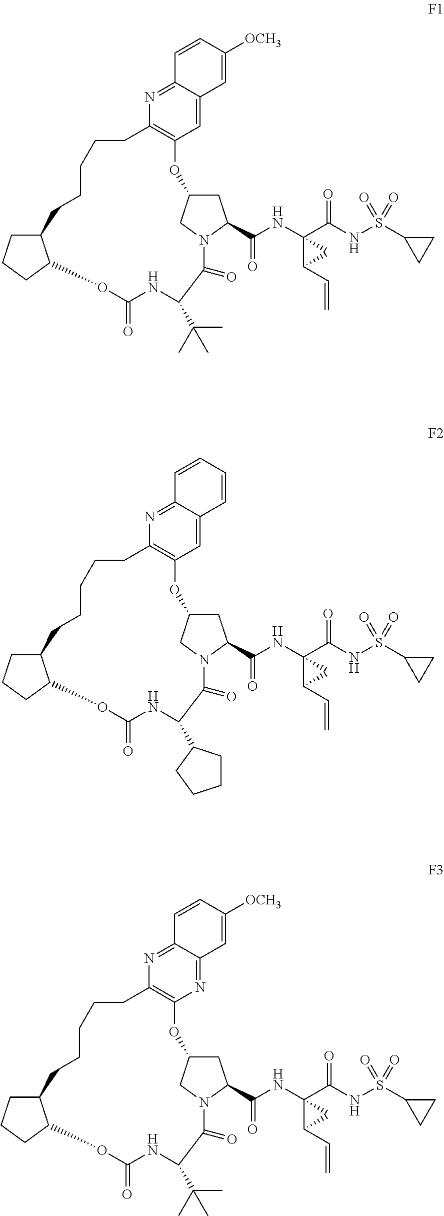

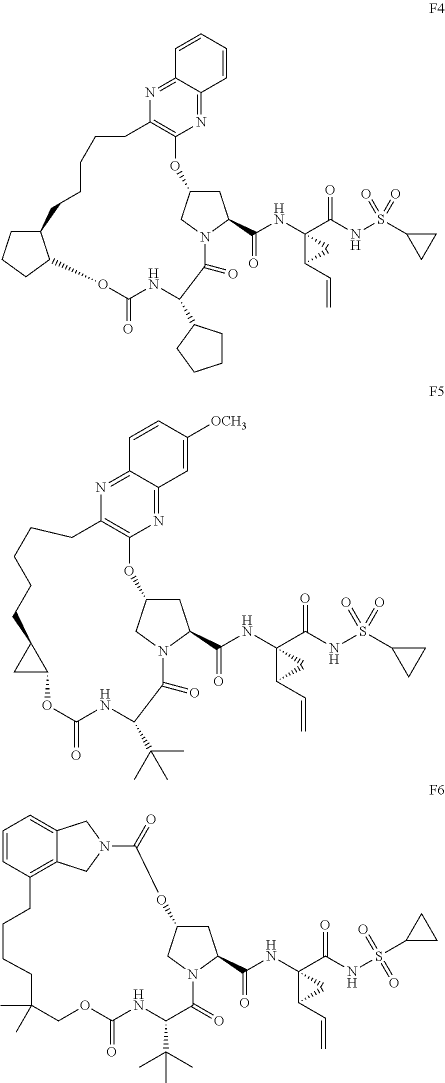

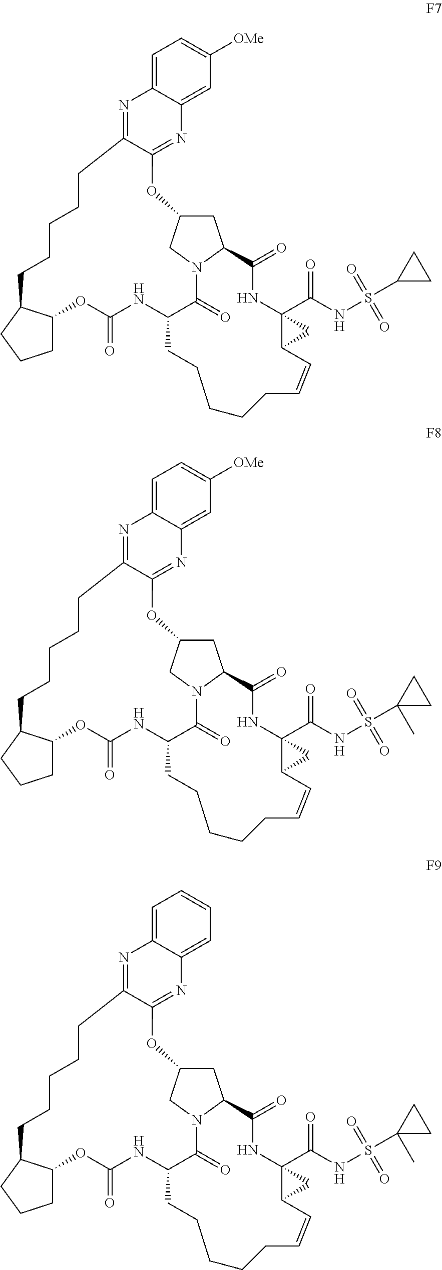

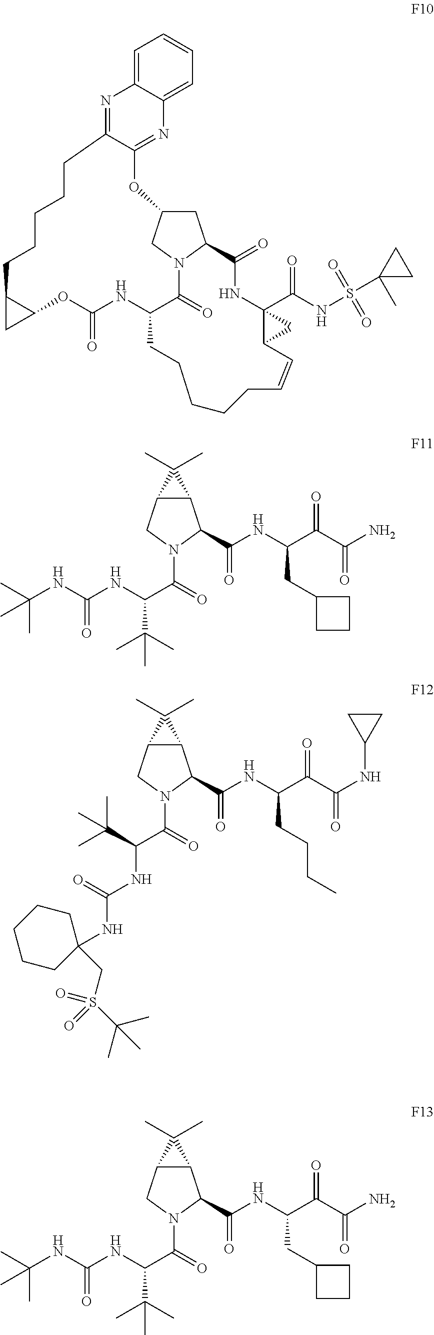

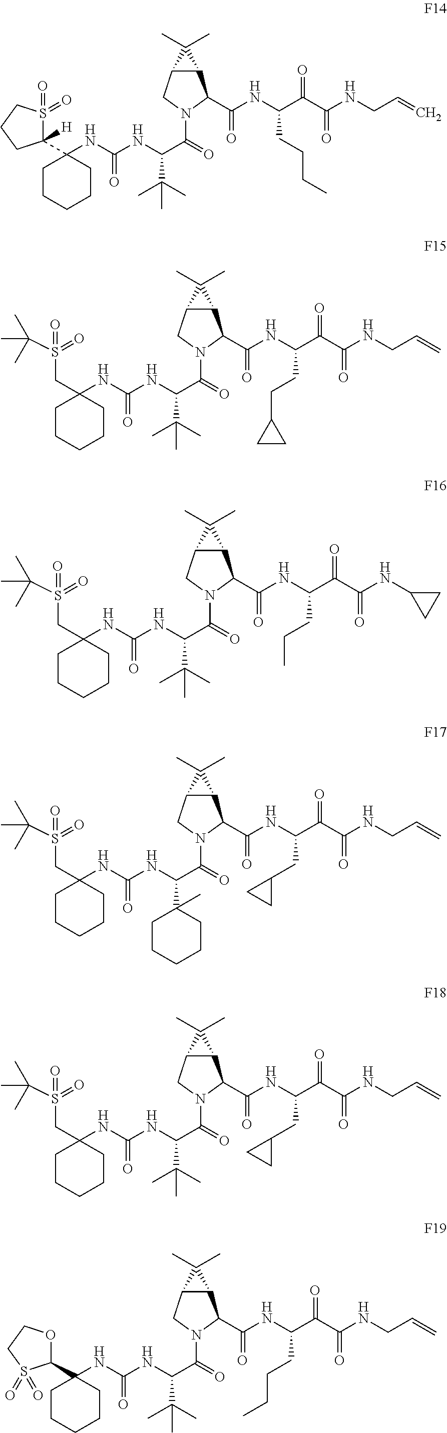

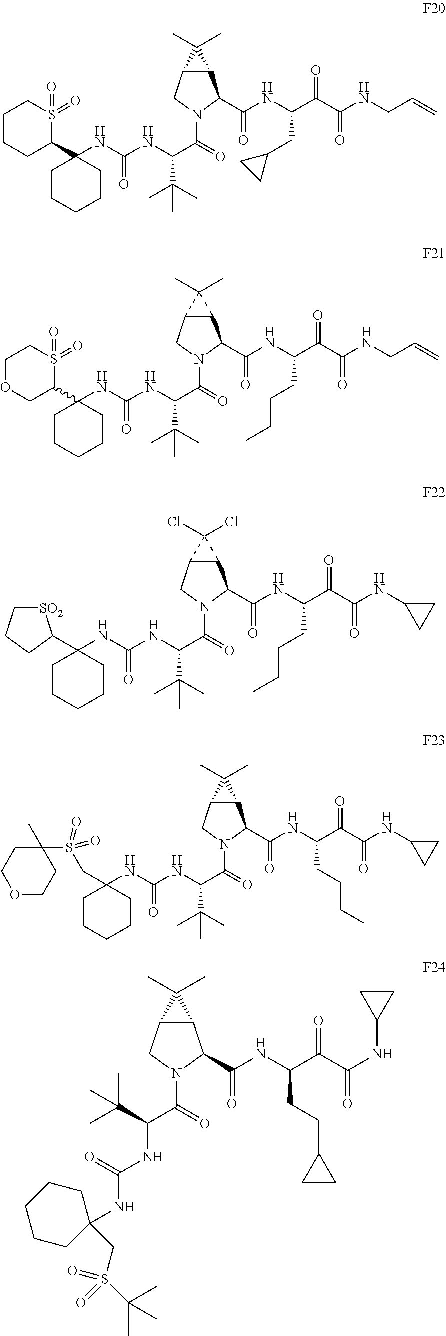

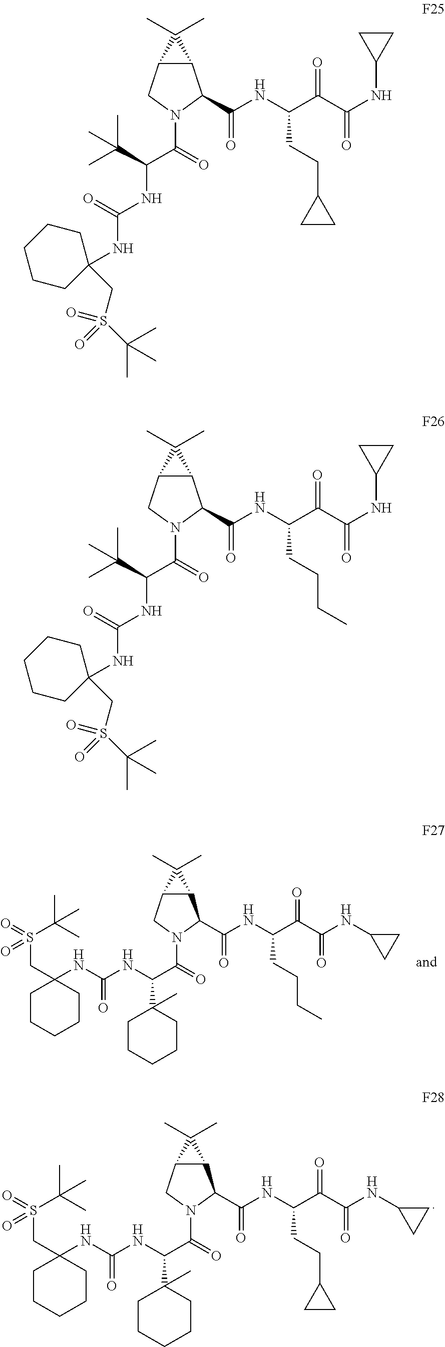 [SMILES: CC(C)(C)[C@H](NC(=O)NC1(CS(=O)(=O)C(C)(C)C)CCCCC1)C(=O)N1C[C@H]2[C@@H]([C@H]1C(=O)N[C@@H](CCC1CC1)C(=O)C(=O)NC1CC1)C2(C)C ; CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1 ; CCCC[C@H](NC(=O)[C@@H]1[C@@H]2[C@H](CN1C(=O)[C@@H](NC(=O)NC1(CS(=O)(=O)C(C)(C)C)CCCCC1)C1(C)CCCCC1)C2(C)C)C(=O)C(=O)NC1CC1 ; CC1([C@H](NC(=O)NC2(CS(=O)(=O)C(C)(C)C)CCCCC2)C(=O)N2C[C@H]3[C@@H]([C@H]2C(=O)N[C@@H](CCC2CC2)C(=O)C(=O)NC2CC2)C3(C)C)CCCCC1]

In one embodiment, for the methods and compositions of the present invention, the first additional therapeutic agent is selected from compounds F5, F6, F7, F11, F13 and F26.

In another embodiment, for the methods and compositions of the present invention, the first additional therapeutic agent is selected from compounds F5 and F7.

### Second Additional Therapeutic Agents

In another embodiment, the present methods for treating or preventing HCV infection comprise the administration of: (i) a Polycyclic Heterocycle Derivative of Table 1; (ii) a first additional therapeutic agent; and (iii) a second additional therapeutic agent.

In one embodiment, agents useful as second additional therapeutic agents in the present compositions and methods are selected from an HCV antiviral agent, an immumodulator and an anti-infective agent.

In another embodiment, agents useful as second additional therapeutic agents in the present compositions and methods are selected from an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor and an antibody therapy (monoclonal or polyclonal).

HCV polymerase inhibitors useful as second additional therapeutic agents in the present compositions and methods include, but are not limited to, BMS-791325 (Bristol-Myers Squibb), VP-19744 (Wyeth/ViroPharma), PSI-7851 (Phannasset), RG7128 (Roche/Pharmasset), PSI-7977 (Pharmasset), PSI-938 (Pharmasset), PSI-879 (Pharmasset), PSI-661 (Pharmasset), PF-868554/filibuvir (Pfizer), VCH-759/VX-759 (ViroChem Pharma/Vertex), HCV-371 (Wyeth/VirroPharma), HCV-796 (Wyeth/ViroPharma), IDX-184 (Idenix), IDX-375 (Idenix), NM-283 (Idenix/Novartis), GL-60667 (Genelabs), JTK-109 (Japan Tobacco), PSI-6130 (Phannasset), R1479 (Roche), R-1626 (Roche), R-7128 (Roche), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), VCH-222/VX-222 (ViroChem/Vertex), VCH-916 (ViroChem), VCH-716(ViroChem), GSK-71185 (Glaxo SmithKline), ANA598 (Anadyr), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews,1:867 (2002); and Beaulieu et al., Current Opinion in Investigational Drugs, 5:838 (2004).

Other HCV polymerase inhibitors useful as second additional therapeutic agents in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125.

Interferons useful as second additional therapeutic agents in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™m Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), R-7025 (Roche), IFN-α-2b-XL (Flamel Technologies), belerofon (Nautilus) and consensus interferon as defined by determination of a consensus sequence of naturally occurring interferon alphas (Infergen™, Amgen, Thousand Oaks, Calif.).

Antibody therapy agents useful as second additional therapeutic agents in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhbitors useful as second additional therapeutic agents in the present compositions and methods include, but are not limited to, an HCV protease inhibitor.

HCV protease inhibitors useful as second additional therapeutic agents in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful as second additional therapeutic agents in the present compositions and methods include, but are not limited to, VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott/Enanta), TMC-435350 (Medivir), RG7227 (Danoprevir, InterMune/Roche), EA-058 (Abbott/Enanta), EA-063 (Abbott/Enanta), GS-9256 (Gilead), IDX-320 (Idenix), ACH-1625 (Achillion), ACH-2684 (Achillion), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhbitors useful as second additional therapeutic agents in the present compositions and methods include, but are not limited to, those disclosed in Landro et al., Biochemistry, 36(31):9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Viral replication inhibitors useful as second additional therapeutic agents in the present compositions and methods include, but are not limited to, HCV replicase inhibitors, IRES inhibitors, NS4A inhibitors, NS3 helicase inhibitors, NS5A inhibitors, NS5B inhibitors, ribavirin, AZD-2836 (Astra Zeneca), viramidine, A-831 (Arrow Therapeutics), EDP-239 (Enanta), ACH-2928 (Achillion), GS-5885 (Gilead); an antisense agent or a therapeutic vaccine.

Viral entry inhibitors useful as second additional therapeutic agents in the present compositions and methods include, but are not limited to, PRO-206 (Progenies), REP-9C (REPICor), SP-30 (Samaritan Pharmaceuticals) and ITX-5061 (iTherx).

HCV NS4A inhibitors useful as second additional therapeutic agents in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful as second additional therapeutic agents in the present compositions and methods include, but are not limited to, AZD2836 (Astra Zeneca), ACH-1095 (Achillion) and ACH-806 (Achillion).

HCV NS5A inhibitors useful as second additional therapeutic agents in the present compositions and methods include, but are not limited to, A-832 (Arrow Therpeutics), PPI-461 (Presidio), PPI-1301 (Presidio) and BMS-790052 (Bristol-Myers Squibb).

HCV replicase inhibitors useful as second additional therapeutic agents in the present compositions and methods include, but are not limited to, those disclosed in U.S. Patent Publication No. US20090081636.

Therapeutic vaccines useful as second additional therapeutic agents in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C (Inovio/Tripep), PeviPRO™ (Pevion Biotect), HCV/MF59 (Chiron/Novartis), MBL-HCV1 (MassBiologics), GI-5005 (Globelmmune), CT-011 (CureTech/Teva) and Civacir (NABI).

Examples of further additional therapeutic agents useful as second additional therapeutic agents in the present compositions and methods include, but are not limited to, Ritonavir (Abbott), TT033 (Benitec/Tacere Bio/Pfizer), Sirna-034 (Sirna Therapeutics), GNI-104 (GENimmune), GI-5005 (Globelmmune), IDX-102 (Idenix), Levovirin™ (ICN Pharmaceuticals, Costa Mesa, Calif.); Humax (Genmab), ITX-2155 (Ithrex/Novartis), PRO 206 (Progenies), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Dehio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., N.C.); Alinia (Romark Laboratories), INFORM-1 (a combination of R7128 and ITMN-191); and mycophenolate mofetil (Hoffman-LaRoche, Nutley, N.J.).

In one embodiment, the second additional therapeutic agent is PSI-7977, RG-7128 or PSI-938.

In another embodiment, the second additional therapeutic agent is PSI-7977.

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the Polycyclic Heterocycle Derivative(s) and the additional therapeutic agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

Generally, a total daily dosage of the Polycyclic Heterocycle Derivatives alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when an additional therapeutic agent is INTRON-A interferon alpha 2b (commercially available from Schering-Plough Corp.), this agent is administered by subcutaneous injection at 3MIU(12 mcg)/0.5 mL/TIW for 24 weeks or 48 weeks for first time treatment.

In another embodiment, when an additional therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when an additional therapeutic agent is ROFERON A interferon alpha 2a (commercially available from Hoffmann-La Roche), this agent is administered by subcutaneous or intramuscular injection at 3MIU(11.1 mcg/mL)/TIW for at least 48 to 52 weeks, or alternatively 6MIU/TIW for 12 weeks followed by 3MIU/TIW for 36 weeks.

In still another embodiment, when an additional therapeutic agent is PEGASUS interferon alpha 2a pegylated (commercially available from Hoffmann-La Roche), this agent is administered by subcutaneous injection at 180 mcg/ImL or 180 mcg/0.5 mL, once a week for at least 24 weeks.

In yet another embodiment, when an additional therapeutic agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment.

In a further embodiment, when an additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In another embodiment, agents useful as second additional therapeutic agents in the present compositions and methods are selected from an HCV protease inhibitor, an interferon and an HCV polymerase inhibitor.

In still another embodiment, agents useful as second additional therapeutic agents in the present compositions and methods are selected from an interferon and an HCV polymerase inhibitor.

In one embodiment, the second additional therapeutic agent is a viral protease inhibitor.

In another embodiment, the second additional therapeutic agent is a viral replication inhibitor.

In another embodiment, the second additional therapeutic agent is an HCV NS3 protease inhibitor.

In still another embodiment, the second additional therapeutic agent is an HCV NS5B polymerase inhibitor.

In another embodiment, the second additional therapeutic agent is a nucleoside inhibitor.

In another embodiment, the second additional therapeutic agent is an interferon.

In yet another embodiment, the second additional therapeutic agent is an HCV replicase inhibitor.

In another embodiment, the second additional therapeutic agent is an antisense agent.

In another embodiment, the second additional therapeutic agent is a therapeutic vaccine.

In a further embodiment, the second additional therapeutic agent is a virion production inhibitor.

In another embodiment, the second additional therapeutic agent is an antibody therapy.

In another embodiment, the second additional therapeutic agent is an HCV NS2 inhibitor.

In still another embodiment, the second additional therapeutic agent is an HCV NS4A inhibitor.

In another embodiment, the second additional therapeutic agent is an HCV NS4B inhibitor.

In another embodiment, the second additional therapeutic agent is an HCV NS5A inhibitor

In yet another embodiment, the second additional therapeutic agent is an HCV NS3 helicase inhibitor.

In another embodiment, the second additional therapeutic agent is an HCV IRES inhibitor.

In another embodiment, the second additional therapeutic agent is an HCV p7 inhibitor.

In a further embodiment, the second additional therapeutic agent is an HCV entry inhibitor.

In another embodiment, the second additional therapeutic agent is an HCV assembly inhibitor.

In one embodiment, the second additional therapeutic agents comprise a viral protease inhibitor and a viral polymerase inhibitor.

In still another embodiment, the second additional therapeutic agents comprise a viral protease inhibitor and an immunomodulatory agent.

In yet another embodiment, the second additional therapeutic agents comprise a polymerase inhibitor and an immunomodulatory agent.

In another embodiment, the second additional therapeutic agents comprise a viral protease inhibitor and a nucleoside.

In another embodiment, the second additional therapeutic agents comprise an immunomodulatory agent and a nucleoside.

In one embodiment, the second additional therapeutic agents comprise an HCV protease inhibitor and an HCV polymerase inhibitor.

In another embodiment, the second additional therapeutic agents comprise a nucleoside and an HCV NS5A inhibitor.

In another embodiment, the second additional therapeutic agents comprise a viral protease inhibitor, an immunomodulatory agent and a nucleoside.

In a further embodiment, the second additional therapeutic agents comprise a viral protease inhibitor, a viral polymerase inhibitor and an immunomodulatory agent.

In another embodiment, the second additional therapeutic agent is pegylated interferon alpha.

In another embodiment, the second additional therapeutic agent is ribavirin.

In still another embodiment, the second additional therapeutic agent is RG-7128, PSI-938 or PSI-7977.

In another embodiment, the second additional therapeutic agent is PSI-7977.

In one embodiment, the second additional therapeutic agent is pegylated interferon alpha and the combination therapy method further comprises administering ribavirin to the patient.

In one embodiment, a Compound of Table 1 is administered with one or more additional therapeutic agents selected from: an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, a Compound of Table 1 is administered with one or more additional therapeutic agents selected from an HCV protease inhibitor, an HCV polymerase inhibitor, an HCV replication inhibitor, a nucleoside, an interferon, a pegylated interferon and ribavirin. The combination therapies can include any combination of these additional therapeutic agents.

In another embodiment, a Compound of Table 1 is administered with a first additional therapeutic agent and a second additional therapeutic agent, wherein the second additional therapeutic agent is selected from an HCV protease inhibitor, an interferon, a pegylated interferon and ribavirin.

In still another embodiment, a Compound of Table 1 is administered with a first additional therapeutic agent and a second additional therapeutic agent, wherein the second additional therapeutic agent is selected from an HCV protease inhibitor, an HCV replication inhibitor, a nucleoside, an interferon, a pegylated interferon and ribavirin.

In another embodiment, a Compound of Table 1 is administered with a first additional therapeutic agent, a second additional therapeutic agent and a third additional therapeutic which is ribavirin.

In another embodiment, a Compound of Table 1 is administered with a first additional therapeutic agent, an interferon and ribavirin.

In yet another embodiment, a Compound of Table 1 is administered with a first additional therapeutic agent, pegylated interferon alpha and ribavirin.

In one embodiment, a Compound of Table 1 is administered with one or more additional therapeutic agents selected from an HCV polymerase inhibitor, a viral protease inhibitor, an interferon, and a viral replication inhibitor. In another embodiment, a Compound of Table 1 is administered with one or more additional therapeutic agents selected from an HCV polymerase inhibitor, a viral protease inhibitor, an interferon, and a viral replication inhibitor. In another embodiment, a Compound of Table 1 is administered with one or more additional therapeutic agents selected from an HCV polymerase inhibitor, a viral protease inhibitor, an interferon, and ribavirin.

In one embodiment, a Compound of Table 1 is administered with one additional therapeutic agent selected from an HCV polymerase inhibitor, a viral protease inhibitor, an interferon, and a viral replication inhibitor. In another embodiment, a Compound of Table 1 is administered with ribavirin.

In one embodiment, a Compound of Table 1 is administered with two additional therapeutic agents selected from an HCV polymerase inhibitor, a viral protease inhibitor, an interferon, and a viral replication inhibitor.

In another embodiment, a Compound of Table 1 is administered with ribavirin, interferon and another therapeutic agent.

In another embodiment, a Compound of Table 1 is administered with ribavirin, interferon and another therapeutic agent, wherein the additional therapeutic agent is selected from an HCV polymerase inhibitor, a viral protease inhibitor, and a viral replication inhibitor.

In still another embodiment, a Compound of Table 1 is administered with ribavirin, interferon and a viral protease inhibitor.

In another embodiment, a Compound of Table 1 is administered with ribavirin, interferon and an HCV protease inhibitor.

In another embodiment, a Compound of Table 1 is administered with ribavirin, interferon and boceprevir or telaprevir.

In a further embodiment, a Compound of Table 1 is administered with ribavirin, interferon and an HCV polymerase inhibitor.

In another embodiment, a Compound of Table 1 is administered with pegylated-interferon alpha and ribavirin.

In one embodiment, a Compound of Table 1 is administered with (i) compound F5 or F7 and (ii) RG-7128, PSI-938 or PSI-7977.

In another embodiment, a Compound of Table 1 is administered with compound F5 and PSI-7977.

### Compositions and Administration

Due to their activity, the Polycyclic Heterocycle Derivatives are useful in veterinary and human medicine. As described above, the Polycyclic Heterocycle Derivatives are useful for treating or preventing HCV infection in a patient in need thereof.

When administered to a patient, the Polycyclic Heterocycle Derivatives can be administered as a component of a composition that comprises a pharmaceutically acceptable carrier or vehicle. The present invention provides pharmaceutical compositions comprising an effective amount of at least one Polycyclic Heterocycle Derivative and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i. e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the Polycyclic Heterocycle Derivatives are administered orally.

In another embodiment, the Polycyclic Heterocycle Derivatives are administered intravenously.

In another embodiment, the Polycyclic Heterocycle Derivatives are administered topically.

In still another embodiment, the Polycyclic Heterocycle Derivatives are administered sublingually.

In one embodiment, a pharmaceutical preparation comprising at least one Polycyclic Heterocycle Derivative is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Polycyclic Heterocycle Derivative(s) by weight or volume. In various embodiments, the present compositions can contain, in one embodiment, from about 1% to about 70% or from about 5% to about 60% of the Polycyclic Heterocycle Derivative(s) by weight or volume.

The quantity of Polycyclic Heterocycle Derivative in a unit dose of preparation may be varied or adjusted from about I mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over,a 24 hour period. In still another embodiment, the total daily dosage is administered in four divided doses over a 24 hour period.

The amount and frequency of administration of the Polycyclic Heterocycle Derivatives will be regulated according to the judgment of the attending clinician considering such factors as age, condition and size of the patient as well as severity of the symptoms being treated. Generally, a total daily dosage of the Polycyclic Heterocycle Derivatives range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Polycyclic Heterocycle Derivative or a pharmaceutically acceptable salt thereof; (ii) one or more additional therapeutic agents that are not a Polycyclic Heterocycle Derivative; and (iii) a pharmaceutically acceptable carrier, wherein the amounts in the composition are together effective to treat HCV infection.

In one embodiment, the present invention provides compositions comprising: (i) a pharmaceutically acceptable carrier; (ii) a Compound of Table 1 or a pharmaceutically acceptable salt thereof; and (iii) a first additional therapeutic agent or a pharmaceutically acceptable salt thereof.

In another embodiment, the present invention provides compositions comprising: (1) a pharmaceutically acceptable carrier; (ii) a Compound of Table 1 or a pharmaceutically acceptable salt thereof; (iii) a first additional therapeutic agent or a pharmaceutically acceptable salt thereof; and (iv) a second additional therapeutic agent or a pharmaceutically acceptable salt thereof.

In one embodiment, the present invention provides compositions comprising: (i) a pharmaceutically acceptable carrier; (ii) a Compound of Table 1 or a pharmaceutically acceptable salt thereof; (iii) a first additional therapeutic agent or a pharmaceutically acceptable salt thereof; and (iv) a second additional therapeutic agent or a pharmaceutically acceptable salt thereof, wherein the second additional therapeutic agent is selected from an HCV antiviral agent, an immunomodulator or an anti-viral agent.

In another embodiment, the present invention provides compositions comprising: (i) a pharmaceutically acceptable carrier; (ii) a Compound of Table 1 or a pharmaceutically acceptable salt thereof; (iii) a first additional therapeutic agent or a pharmaceutically acceptable salt thereof; and (iv) a second additional therapeutic agent or a pharmaceutically acceptable salt thereof, wherein the second additional therapeutic agent is selected from an HCV polymerase inhibitor, an interferon or an HCV protease inhibitor.

In another embodiment, the present invention provides compositions comprising: (i) a pharmaceutically acceptable carrier; (ii) a Compound of Table 1 or a pharmaceutically acceptable salt thereof; (iii) a first additional therapeutic agent or a pharmaceutically acceptable salt thereof; and (iv) a second additional therapeutic agent or a pharmaceutically acceptable salt thereof, wherein the second additional therapeutic agent is selected from an HCV polymerase inhibitor and an interferon.

In still another embodiment, the present invention provides compositions comprising: (i) a pharmaceutically acceptable carrier; (ii) a Compound of Table 1 or a pharmaceutically acceptable salt thereof; (iii) a first additional therapeutic agent or a pharmaceutically acceptable salt thereof; and (iv) a second additional therapeutic agent or a pharmaceutically acceptable salt thereof, wherein the second additional therapeutic agent is selected from ribavirin and pegylated interferon alpha.

In another embodiment, the present invention provides compositions comprising: (i) a pharmaceutically acceptable carrier; (ii) a Compound of Table 1 or a pharmaceutically acceptable salt thereof; (iii) a first additional therapeutic agent or a pharmaceutically acceptable salt thereof; (iv) ribavirin; and (v) pegylated interferon alpha.

### Kits

In one aspect, the present invention provides a kit comprising: (i) a Polycyclic Heterocycle Derivative or a pharmaceutically acceptable salt thereof and (ii) a first additional therapeutic agent or a pharmaceutically acceptable salt thereof, wherein the amounts of the two active ingredients together result in a desired therapeutic effect. In one embodiment, the Polycyclic Heterocycle Derivative and the first additional therapeutic agents are provided in the same container. In another embodiment, the Polycyclic Heterocycle Derivative and the first additional therapeutic agents are each provided in separate container.

In another aspect, the present invention provides a kit comprising: (i) a Polycyclic Heterocycle Derivative or a pharmaceutically acceptable salt thereof; (ii) a first additional therapeutic agent or a pharmaceutically acceptable salt thereof; and (iii) a second additional therapeutic agent or a pharmaceutically acceptable salt thereof, wherein the amounts of the three active ingredients together result in a desired therapeutic effect. In one embodiment, the Polycyclic Heterocycle Derivative and the first additional therapeutic gents are provided in the same container. In another embodiment, the Polycyclic Heterocycle Derivative, the first additional therapeutic agent and the second additional therapeutic agent are each provided in a separate container.

### EXAMPLES

### Example 1

### Preparation of the Polycyclic Heterocycle Derivatives of Table 1 and Additional Therapeutic Agents F1-F28

The compounds of Table 1 can be made as described in International Publication Nos. WO 10/111483 and and International Application No. PCT/US2011/027117, each of which are incorporated herein by reference in their entirety.

Alternatively, it will be obvious to one skilled in the art of organic synthesis how to make the compounds of Table 1 using the methods described in “Comprehensive Heterocyclic Chemistry” editions I, II and III, published by Elsevier and edited by A. R. Katritzky & R. J K Taylor; US Patent Publication No. US20080050336; and International Publication No. WO 10/065674.

The additional therapeutic agents F1-F28 can be made, for example, using the methods described in U.S. Patent Publication No. US 2010/0099695 and U.S. Pat. Nos. 7,012,066, 7,244,721, 7,470,664 and 7,973,040, each of which are incorporated herein by reference in their entirety.

### Example 2

### Procedure for Combination Studies

The synergy of compounds of the present invention in combination with an additional therapeutic agent can be measured using the combination study described below.

This cell-based in vitro combination study is performed using a 384 well plate, which is divided into 4 quadrants (for quadruplicate determination). A 9 point horizontal (2 fold dilution) titration for the first test compound and a 6 point vertical (2 fold dilution) titration of the second test compound are placed in each quadrant. In another 384 well plate, the opposite orientation is tested: the second test compound is diluted in 9 point titration, 2-fold and the first test compound is diluted in 6 point titration, 2-fold.

Note: The EC₅₀ for each individual test compound is to be determined prior to initiation of the in vitro combination assay using the RHEPLUC assay described below. The previously determined EC₅₀ for each test compound is then placed in the middle of the combination study titration curve.

### RHEPLUC Assay

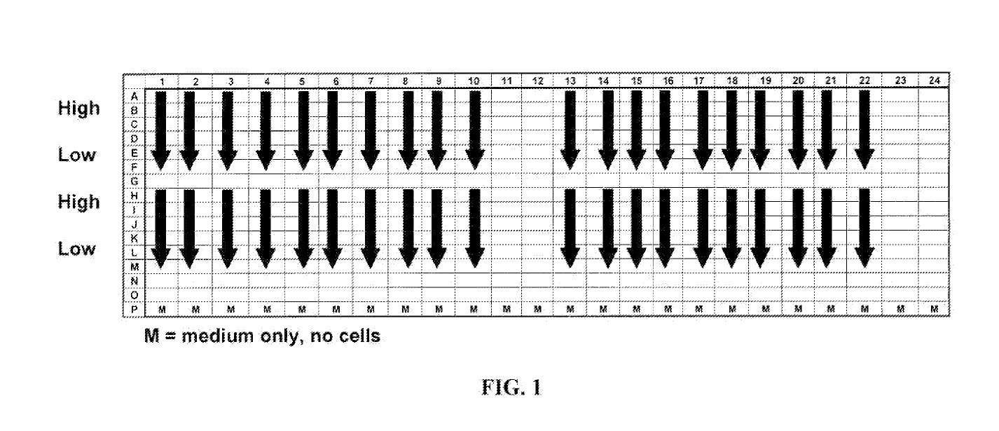

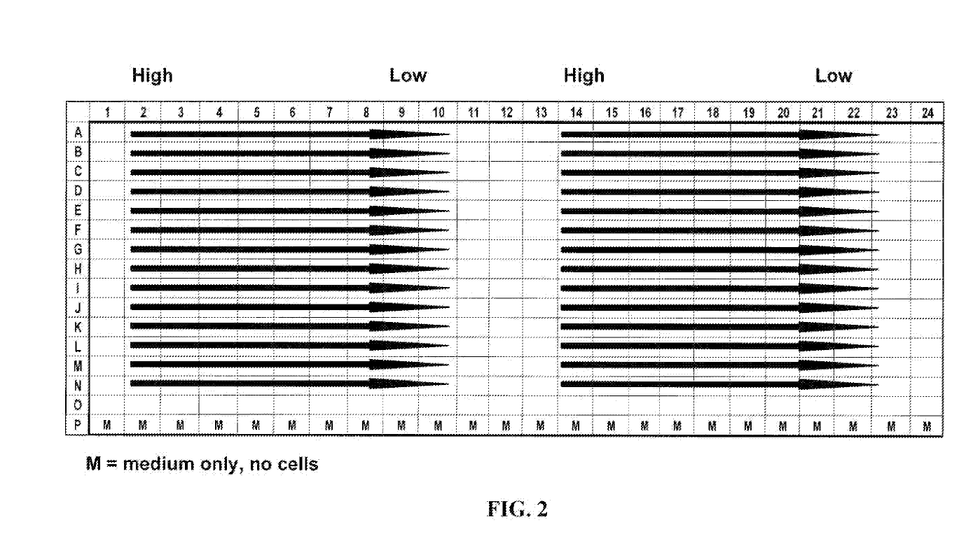

Test compounds are ordered at 4000× final concentration, one or two days before the assay, and are diluted 1/10 in DMSO (400× concentration). The diluted first test compound (400×) is distributed in a first low dead volume plate as described in FIG. 1. The diluted second test compound (400×) is distributed in a second low dead volume plate as described in FIG. 2.

The first and second low dead volume plates are mixed together by transferring 3 μL of each plate into a third low dead volume plate (referred to herein as the “200× test compounds mix plate”).

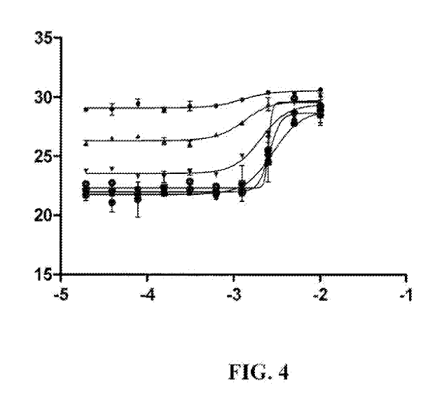

To obtain maximum luciferase signal (0% inhibition control), 100% DMSO is added to the 200× test compound mix plate. For minimum luciferase signal (100% inhibition control), a known NS5A inhibitor (Compound A, EC₅₀ of 0.01 nM, made as described in International Patent Application No. PCT/US2010/028653) is used at 1 nM final concentration. Accordingly, 6 μL of 200 nM Compound A is added to the 200× test compound mix plate (see FIG. 4).

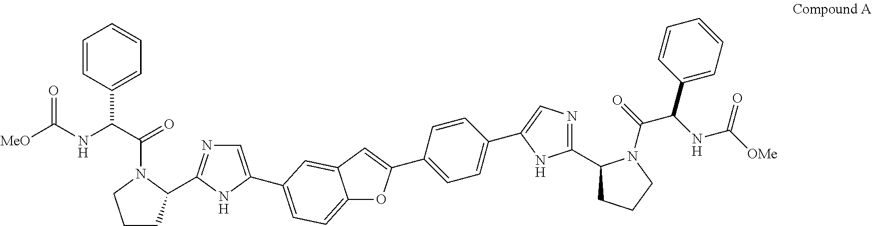

The 200× test compounds mix plate is then stored in a dessicator at room temperature until needed.

### Preparation of Compound Plate

On the day of the experiment, Complete growth media (10 μL) is added to all wells in a 384 well assay plate, then 150 nL per well from the test compound mix plate is added to the assay plate.

### Cell Preparation

The cell monolayer is rinsed with pre-warmed PBS (˜37° C.), then pre-warmed trypsin (0.25%, ˜37° C.) is added and the cells are incubated for 2 to 5 minutes in 5% CO₂ at 37° C. Complete growth media is then added, cells are mixed and counted, and then diluted in complete growth media to a final concentration of 1.0×10⁵ cells/mL. The cells are then filtered using a 70 μm cell strainer.

### Addition of Test Compounds to Cells

20 μL of cells/well are added to the assay plate containing 10 μL of complete growth media and 150 nL of test compound (as prepared above) to provide 30 μL total (final concentration of DMSO is 0.5% with 2000 cells/well).

The plate is incubated for 30 minutes at room temperature, then the plate is transferred to an incubator and incubated for 72 hours in 5% CO₂ at 37° C.

### Detection

Bright-Glo Luciferase reagent is prepared as specified by the kit instructions and kept in the dark until the reagent has reached room temperature. The cell incubated plate is then taken out of the incubator and allowed to equilibrate at room temperature for 30 minutes, after which time 30 μL of the prepared Bright-Glo Luciferase reagent is added to the cell incubated plate. The plate is allowed to incubate for 5 minutes at room temperature, then the plate is placed on a reader within 30 minutes after incubation is complete, and the luminescence is monitored at 0.5 seconds per well.

### Analysis

The combination study data can be analyzed using MacSynergy software and CompuSyn software according to the respective user's guides.

Using the RHEPLUC assay, EC₅₀ values were calculated prior to initiation of combination studies for a selected Polycyclic Heterocycle Derivative of the present invention (compound 2) and for two first additional therapeutic agents of the present invention (compounds F5 and F7). The results are set forth in the table below.

| | EC₅₀ |
|---|---|
| Compound | (nM) |
| 2 | 0.004 |
| F5 | 0.3215 |
| F7 | 0.55 |

Note:
In parallel to the combination studies, a cytotoxicity experiment is carried out in order to measure cell toxicity and to ensure that the inhibition of replication seen is not due to cytotoxicity. The protocol used is described below in Example 4.

### Example 3

### Synergy Determination for Combination Therapies of the Present Invention

### Combination of Compound 2 and the First Additional Therapeutic Agent Compound F5

Using the Combination Study protocol described in Example 2, the combination of (i) Compound 2 of Table 1 and (ii) Compound F5 was tested. Data was analyzed using MacSynergy software and the results are set forth in the table below, wherein a log volume of <2 indicates no synergy, a log volume of 2-5 indicates minor but significant synergy, a log volume of 5-9 indicates moderate synergy, a log volume of >9 indicates strong synergy, and a log volume of >90 indicates unreliable data.

### Replicates

These results indicate that the combination of Compound 2 and Compound F5 demonstrates moderate to strong synergy in vitro and suggests that this particular combination will be synergistic in vivo.

Combination of the first therapeutic agent

99.9% confidence

| | 1 | 2 | 3 | 4 | 5 | 6 |
|---|---|---|---|---|---|---|
| Synergy | 186 | 132 | 53 | 61 | 40 | 84 |
| Log volume | 17 | 12 | 5 | 6 | 4 | 8 |

Compound 2 and additional Compound F7

Using the Combination Study protocol described in Example 2, the combination of Compound 2 and Compound F7 was tested. Data was analyzed using MacSynergy software and the results are set forth in the table below, wherein a log volume of <2 indicates no synergy, a log volume of 2-5 indicates minor but significant synergy, a log volume of 5-9 indicates moderate synergy, a log volume of >9 indicates strong synergy, and a log volume of >90 indicates unreliable data.

### Replicates

99.9% confidence

| | 1 | 2 | 3 | 4 | 5 | 6 |
|---|---|---|---|---|---|---|
| Synergy | 129 | 80 | 87 | 140 | 57 | 59 |
| Log volume | 12 | 7 | 8 | 13 | 5 | 5 |

These results indicate that the combination of Compound 2 and Compound F7 demonstrates moderate to strong synergy in vitro and suggests that this particular combination will be synergistic in vivo.

### Example 4

### Determination of Cytotoxicity

The cytotoxicity of compounds of Table 1 and of the additional therapeutic agents used in the compositions and methods of the present invention can be measure using the assay described below.

### Compound and Cell Preparation

Compounds are ordered 1 or 2 days before the experiment. Compounds and cells are prepared using the method described in Example 2 for the RHEPLUC assay (See FIG. 1) or combination study (See FIG. 2)

After 3 day incubation of cells with individual test compounds, cytotoxicity is determined using CellTiter Blue as described in the assay protocol below.

### Cytotoxicity Assay

The CellTiter Blue solution (4 mL) is diluted with 1× Dulbecco's PBS (16 mL). 5 μL of the resulting solution is added to each well of the 384 well assay plate containing cells treated with compound for 72 hours. The plate is shaken for 10 seconds, then incubated in 5% CO₂ at 37° C. for 1 hour. The plate is then shaken again for 10 seconds and the fluorescence is measured at excitation wavelength 540 nm and emission wavelength 590 nm.

NOTE: cells (all genotypes and mutants) are cultured in DMEM/10% FBS in the presence of G418. During the assay G418 is absent.

### Analysis

For cytotoxicity assays data is analyzed to obtain the CC₅₀ of each compound tested alone and in combination. The analysis uses the average of 100% cytotoxicity (media only without cells) and 0% cytotoxicity (100% viability, 0.5% DMSO in the presence of cells) to calculate the percentage of compound cytotoxicity and CC₅₀ using the following 4 parameter equation:

$$1 - ( \frac{sample - average low}{average high - average low} ) \times 100$$

Wherein average low=100% cytotoxicity (media only) and average high=100% viability (0.5% DMSO).

CC₅₀ values were calculated for a selected Polycyclic Heterocycle Derivative of the present invention (compound 2) and for three first additional therapeutic agents of the present invention. The therapeutic index for the selected compounds was also calculated, wherein TI=CC50/EC₅₀. The results are set forth in the table below.

| | EC₅₀ | CC₅₀ | Therapeutic |
|---|---|---|---|
| Compound | (nM) | (nM) | Index |
| 2 | 0.004 | 9700 | 2425000 |
| F5 | 0.3215 | 35000 | 108865 |
| F7 | 0.55 | 11500 | 20909 |

All compounds tested demonstrate high antiviral activity with minimal cell toxicity. Accordingly, all have high therapeutic indexes and, as such, the synergy data set forth in Example 3 is due to the antiviral activity of the test compounds and not impacted by cytotoxicity.

### Example 5

### 3-Day Cell-Based HCV Replicon Assay

To measure cell-based anti-HCV activity of selected compounds of the present invention, replicon cells were seeded at 3500 cells/well in 96-well collagen I-coated Nunc plates in the presence of the test compound. Various concentrations of test compound, typically in 10 serial 2-fold dilutions, were added to the assay mixture, with the starting concentration ranging from 10 μM to 1 nM. The final concentration of DMSO was 0.5%, fetal bovine serum was 5%, in the assay media. Cells were harvested on day 3 by the addition of 1× cell lysis buffer (Ambion cat 48721). The replicon RNA level was measured using real time PCR (Taqman assay). The PCR primers for gt 1b replicon were: 5B.2F, ATGGACAGGCGCCCTGA (SEQ ID NO. 1); 5B.2R, TTGATGGGCAGCTTGGTTTC (SEQ ID NO. 2); the probe sequence was FAM-labeled CACGCCATGCGCTGCGG (SEQ ID NO. 3). The PCR primers for gt 1a replicon were 5′ primer TGCGGAACCGGTGAGTACA (SEQ ID NO. 4), 3′ primer CGGGTTTATCCAAGAAAGGA (SEQ ID NO. 5) and probe 6FAM-CGGAATTGCCAGGACGACCGG (SEQ ID NO. 6)-TAMRA. The real-time RT-PCR reactions were nm on ABI PRISM 7900HT Sequence Detection System using the following program: 48° C. for 30 minutes, 95° C. for 10 minutes, 40 cycles of 95° C. for 15 sec, 60° C. for 1 minutes. The CT values (cycle of threshold) were plotted against the concentration of test compound and fitted to the sigmoid dose-response model using XLfit4 (MDL). EC₅₀ was defined as the concentration of inhibitor necessary to achieve ΔCT=1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All Taqman reagents were from PE Applied Biosystems. Such an assay procedure was described in detail in e.g. Malcolm et al., Antimicrobial Agents and Chemotherapy 50: 1013-1020 (2006).

### Example 6

### 15-Day Cell-Based HCV Replicon Curing Assay

Using the method described in Example 5, genotype 1a replicon cells were seeded in 6 well plates and dosed with Compound 2 at 0.006 nM (1×EC₉₀) and Compound F5 at 7.5 nM (3×EC₉₀), respectively, and in combination in the presence of 0.5% DMSO. Cell samples were taken at 0, 8, 32, 56 hours followed by 3.5, 8, 11 and 15 days. Total RNA was isolated from cell pellet and HCV RNA was measured using Taqman analysis and normalized by GAPDH RNA.

### Example 7

### Short Term Determination of Inhibition for Combination

Using the 3-day HCV replicon assay described in Example 5, the inhibitory activity of Compound 2 and Compound F5, was determined alone and in combination. Briefly, Genotype 1a replicon cells were dosed with 10 points 2-fold titrations of Compound 2 staring with 0.01 nM horizontally across the plate and with 10 points 2-fold titrations of Compound F5 staring with 5 nM vertically across the plate. Compound 2 and Compound F5 were also titrated as single agents in the absence of the other inhibitor. HCV RNA levels were quantified using the 3-day replicon assay described above in Example 5.

Data was analyzed using Prism and the results are set forth in FIG. 4, which clearly shows that addition of Compound F5 to low concentrations of Compound 2 increased inhibitiory activity and reached maximal inhibition together with high concentrations of Compound 2, demonstrating an additive effect in potency for the combination of Compound 2 and Compound F5.

### Example 8

### Long Term Determination of Inhibition for Combination

Using the 15-day HCV replicon assay, the inhibitory activity of Compound 2 and Compound F5, was determined alone and in combination. Briefly; genotype 1a replicon cells were treated for 15 days with Compound 2 and Compound F5, alone or in combination, using the 15 day HCV replicon assay described in Example 6. Population sequence analysis of NS5A (amino acid residues 1-100) from samples collected at various time points revealed no changes within NS5A. Similar analysis of NS3 (amino acid residues 1-180) from cells treated with Compound F5 showed low levels of D168G and P88A. D168G is known to confer protease resistance, while P88A has not previously been observed.

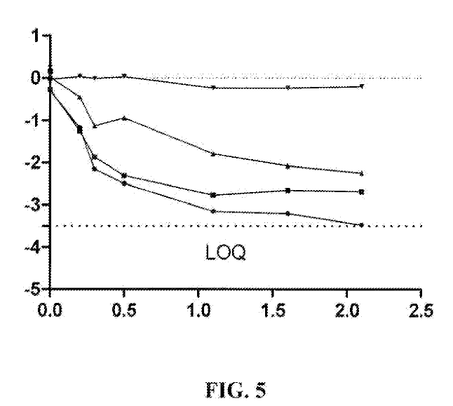

As shown in FIG. 5, The combination of Compound 2 and Compound F5 produced a >3 log RNA reduction to the level of detection, greater than Compound F5 and Compound 2 alone, indicating an additive effect and a lack of antagonism effects. The combination of Compound 2 and Compound F5, however, did not elicit the D168G protease mutation, providing further evidence for the effectiveness of Compound 2 to suppress resistance to other agents when used in combination. Low levels of P88A were still observed in combination and may be due to genetic drift. The combination of Compound 2 and Compound F5 showed increased inhibition of replication in genotype 1a replicon cells to each agent alone.

### Example 9

### Determination of Suppression of Emergence of Resistance

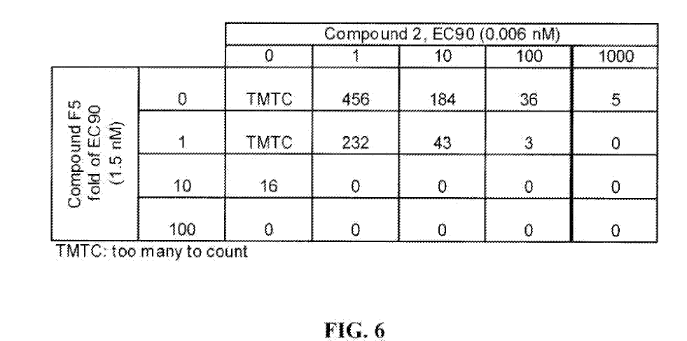

To further examine the effects of Compound 2 and Compound F5 in combination on emergence of resistance, genotype la replicon cells were treated with Compound 2 and Compound F5 in combination under G418 selection, using the 3-day replicon assay described in Example 5. Briefly, the genotype 1a replicon cells were seeded on 60 mm plates at 200,000/plate and were dosed with Compound 2 and Compound F5 in the presence of 0.5% DMSO and 0.5 mg/mL G418 as described in FIG. 6. Cells were split 1:10 three days after dosing and were incubated with test compounds and 0.5 mg/mL G418 for 5 weeks. Cells with DMSO and no test compound were split 1:3 every 3 days. Colonies were then stained and counted visually.

Cells that are free of HCV RNA or contain extremely low levels of HCV RNA are killed by G418 selection, and cells that contain HCV RNA replication that is resistant to Compound 2 and Compound F5 form colonies, which are then counted visually. As FIG. 6 indicates, each test compound by itself reduced resistance frequency in a dose dependent manner. The combination suppressed emergence of resistant colonies to a level that was below detection. Greater reductions in colony count were seen with combinations comparing to Compound F5 and Compound 2 alone, demonstrating an additive effect for this combination in reducing emergence of resistance.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A pharmaceutical composition comprising: (i) a pharmaceutically acceptable carrier; (ii) a compound selected from the following table:
TABLE 1 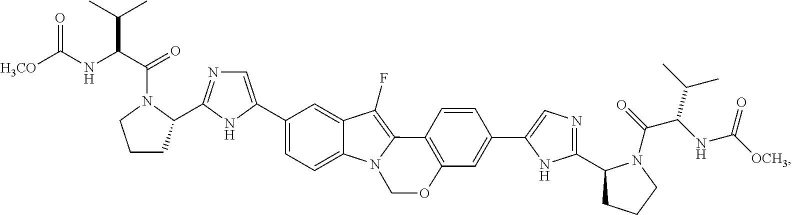 [SMILES: COC(=O)N[C@H](C(=O)N1CCC[C@H]1c1ncc(-c2ccc3c(c2)OCn2c-3c(F)c3cc(-c4cnc([C@@H]5CCCN5C(=O)[C@@H](NC(=O)OC)C(C)C)[nH]4)ccc32)[nH]1)C(C)C] 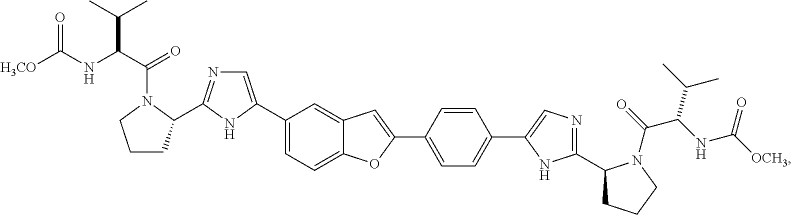 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C(C)C)[nH]5)ccc4o3)cc2)[nH]1)C(C)C] 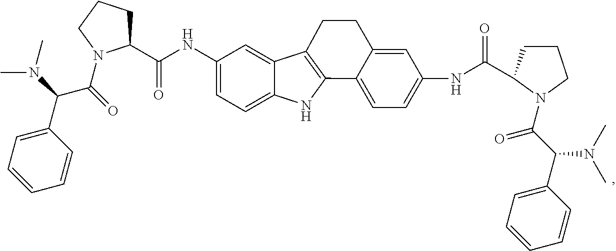 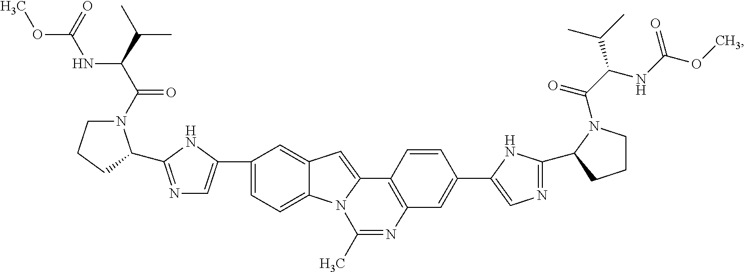 [SMILES: COC(=O)N[C@H](C(=O)N1CCC[C@H]1c1ncc(-c2ccc3c(c2)cc2c4ccc(-c5cnc([C@@H]6CCCN6C(=O)[C@@H](NC(=O)OC)C(C)C)[nH]5)cc4nc(C)n32)[nH]1)C(C)C] 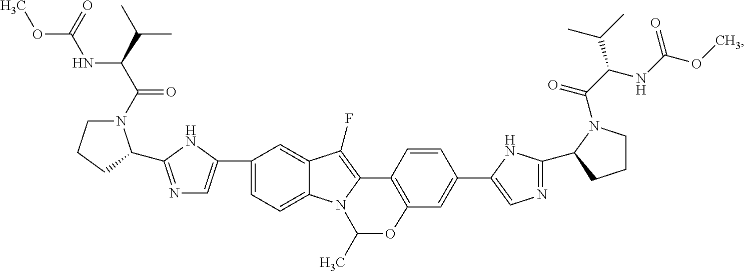 [SMILES: COC(=O)N[C@H](C(=O)N1CCC[C@H]1c1ncc(-c2ccc3c(c2)OC(C)n2c-3c(F)c3cc(-c4cnc([C@@H]5CCCN5C(=O)[C@@H](NC(=O)OC)C(C)C)[nH]4)ccc32)[nH]1)C(C)C] 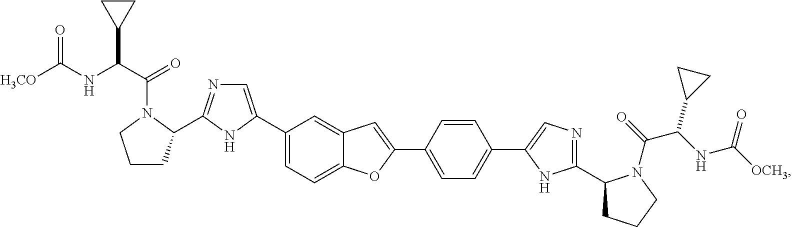 [SMILES: COC(=O)N[C@H](C(=O)N1CCC[C@H]1c1ncc(-c2ccc(-c3cc4cc(-c5cnc([C@@H]6CCCN6C(=O)[C@@H](NC(=O)OC)C6CC6)[nH]5)ccc4o3)cc2)[nH]1)C1CC1] 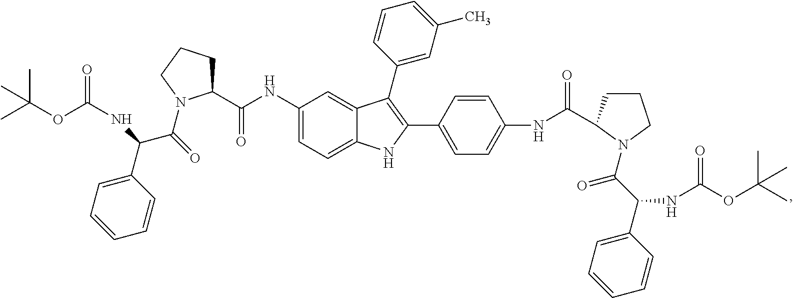 [SMILES: Cc1cccc(-c2c(-c3ccc(NC(=O)[C@@H]4CCCN4C(=O)[C@H](NC(=O)OC(C)(C)C)c4ccccc4)cc3)[nH]c3ccc(NC(=O)[C@@H]4CCCN4C(=O)[C@H](NC(=O)OC(C)(C)C)c4ccccc4)cc23)c1] 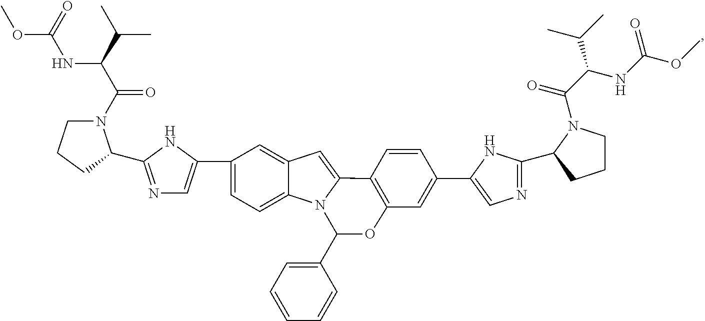 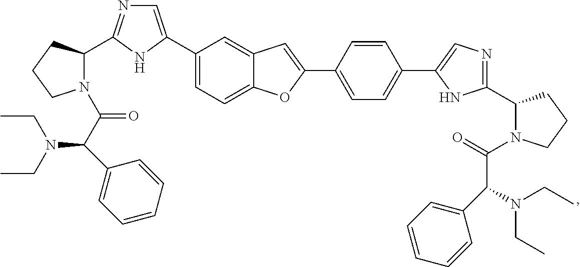 [SMILES: CCN(CC)[C@@H](C(=O)N1CCC[C@H]1c1ncc(-c2ccc(-c3cc4cc(-c5cnc([C@@H]6CCCN6C(=O)[C@@H](c6ccccc6)N(CC)CC)[nH]5)ccc4o3)cc2)[nH]1)c1ccccc1] 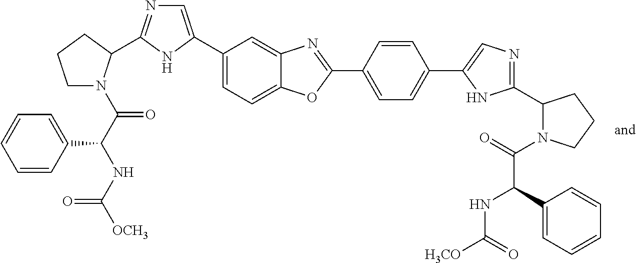 [SMILES: COC(=O)N[C@@H](C(=O)N1CCCC1c1ncc(-c2ccc(-c3nc4cc(-c5cnc(C6CCCN6C(=O)[C@H](NC(=O)OC)c6ccccc6)[nH]5)ccc4o3)cc2)[nH]1)c1ccccc1] 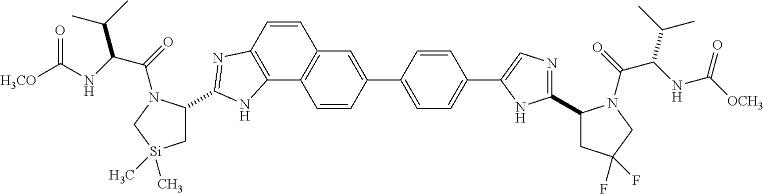
or pharmaceutically acceptable salt thereof; and (iii) a first additional therapeutic agent that is selected from compounds F1-F28, or a pharmaceutically acceptable salt thereof, wherein the amounts of the compound of Table 1 and the first additional therapeutic agent are together effective to treat HCV infection in a patient.

2. The pharmaceutical composition of claim 1, wherein the first additional therapeutic agent is selected from:

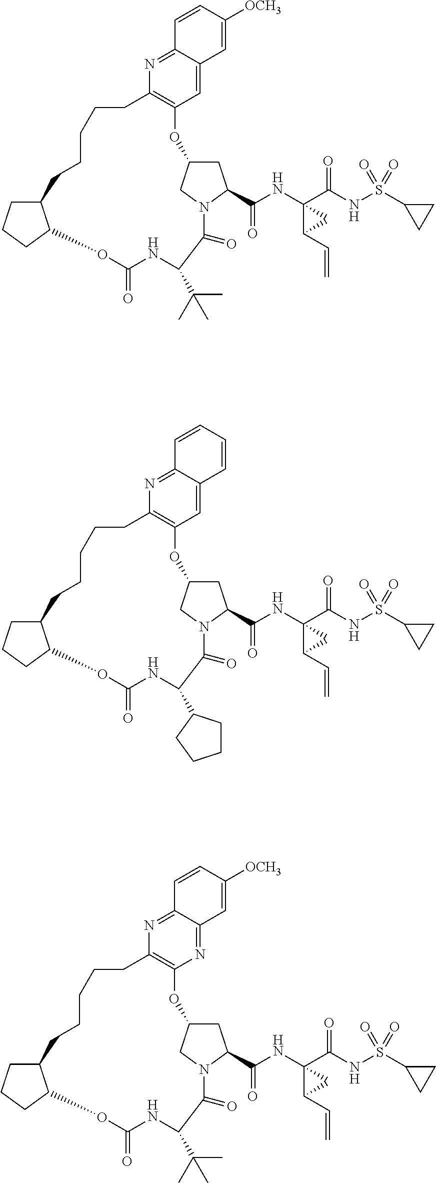

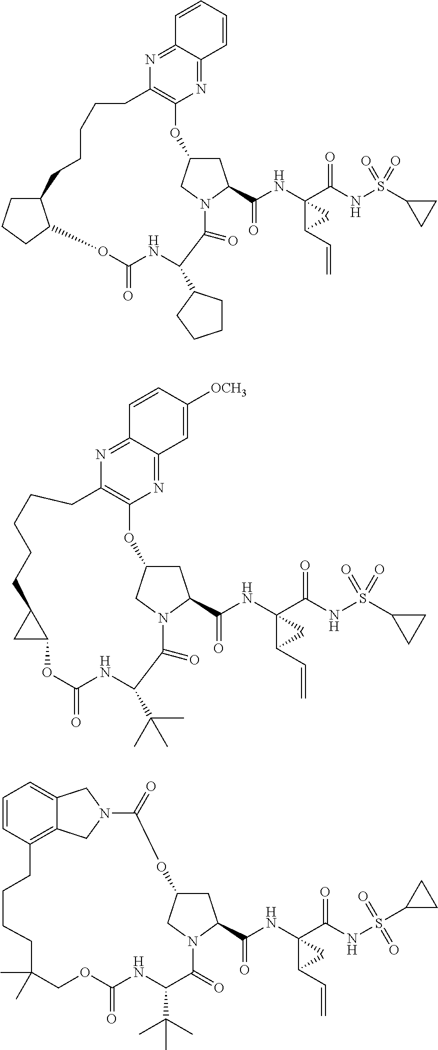

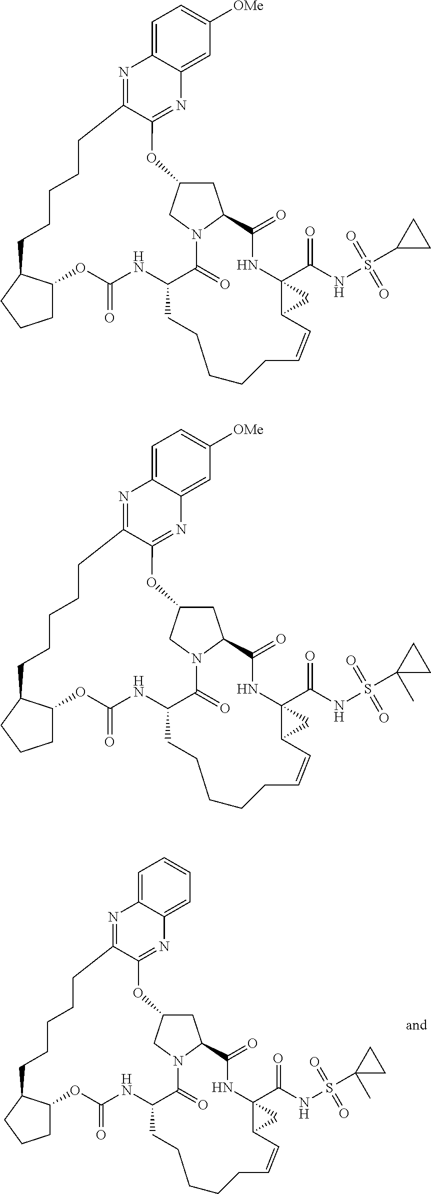

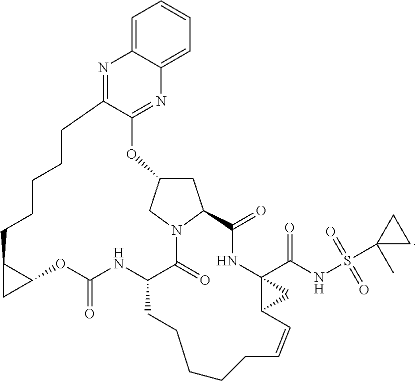 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1]

3. The pharmaceutical composition of claim 2, wherein the first additional therapeutic agent is:

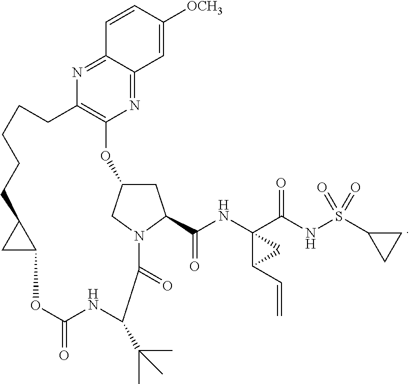 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1]

4. The pharmaceutical composition of claim 1 further comprising a second additional therapeutic agent that is not a compound of Table 1 of claim 1, or a pharmaceutically acceptable salt thereof, wherein the second additional therapeutic agent is selected from an HCV antiviral agent, an immunomodulator and an anti-infective agent.

5. The pharmaceutical composition of claim 4, wherein the second additional therapeutic agent, is selected from an HCV protease inhibitor, an interferon and an HCV polymerase inhibitor.

6. The pharmaceutical composition of claim 5, wherein the second additional therapeutic agent is pegylated interferon alpha.

7. The pharmaceutical composition of claim 6, further comprising ribavirin.

8. A method of treating a patient infected with HCV, the method comprising administering to the patient: (i) a compound selected from Table 1 of claim 1 or a pharmaceutically acceptable salt thereof, and (ii) a first additional therapeutic agent that is selected from compounds F1-F28 or a pharmaceutically acceptable salt thereof, wherein the amounts administered of the compound of Table 1 of claim 1 and the first additional therapeutic agent are together effective to treat the HCV infection.

9. The method of claim 8, wherein the first additional therapeutic agent is selected from:

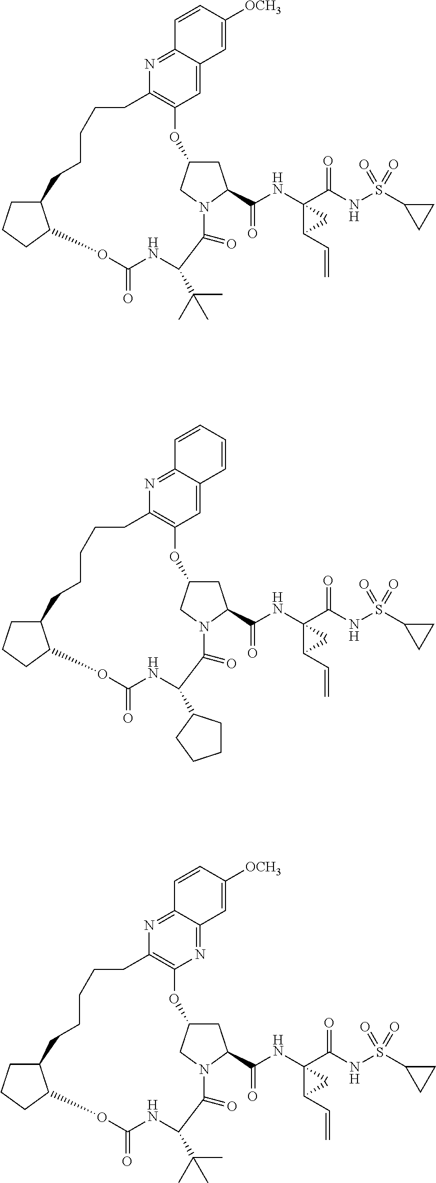

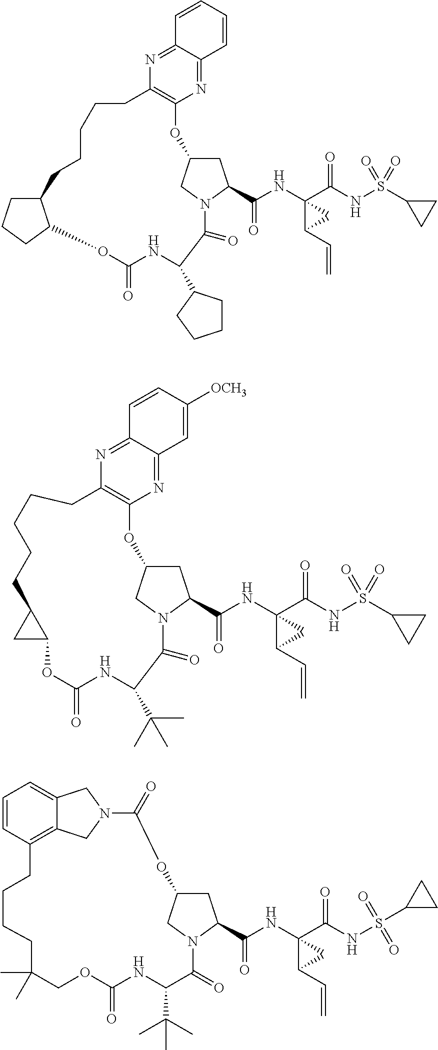

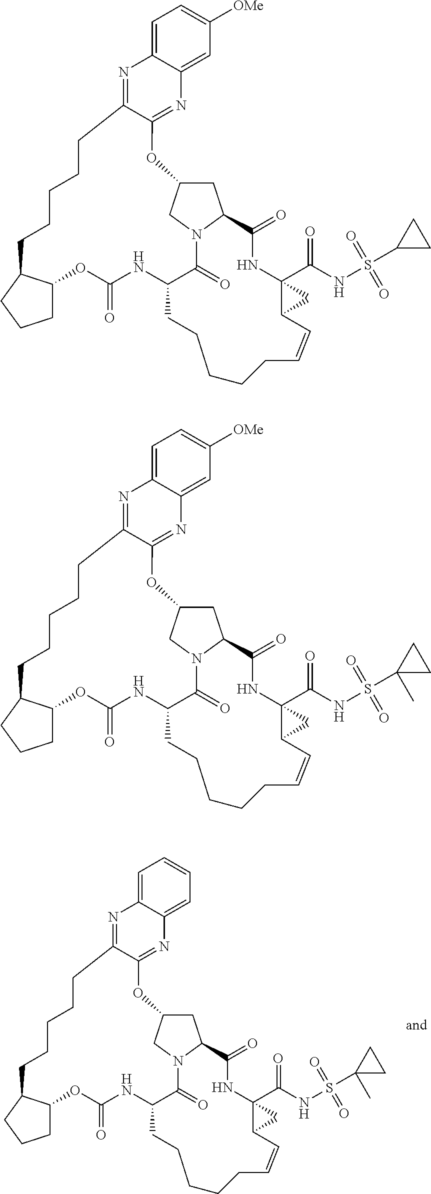

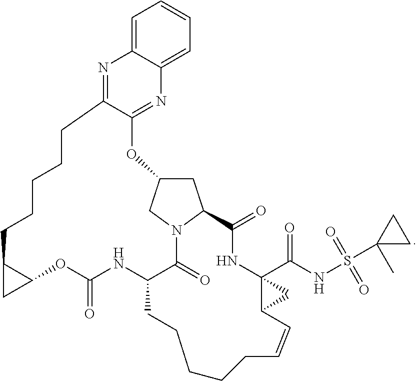 [SMILES: CC1(S(=O)(=O)NC(=O)[C@@]23C[C@H]2/C=C\CCCCC[C@@H]2NC(=O)O[C@@H]4C[C@H]4CCCCCc4nc5ccccc5nc4O[C@@H]4C[C@@H](C(=O)N3)N(C4)C2=O)CC1]

10. The method of claim 8, further comprising administering to the patient a second additional therapeutic agent or a pharmaceutically acceptable salt thereof, wherein the second additional therapeutic agent is selected from an HCV antiviral agent, an immunomodulator and an anti-infective agent.

11. The method of claim 10, wherein the second additional therapeutic agent is selected from an HCV protease inhibitor, an interferon and an HCV polymerase inhibitor.

12. The method of claim 11, wherein the second additional therapeutic agent is pegylated interferon alpha.

13. The method of claim 12, further comprising administering ribavirin to the patient.

14. A method of treating a patient infected with HCV, the method comprising administering to the patient the composition of claim 1.

15. (canceled)


SEQUENCE LISTING (6 sequences)

SEQ ID NO: 1 - DNA, 17 bp, Artificial Sequence
  5B.2F Primer
atggacaggcgccctga

SEQ ID NO: 2 - DNA, 20 bp, Artificial Sequence
  5B.2R Primer
ttgatgggcagcttggtttc

SEQ ID NO: 3 - DNA, 17 bp, Artificial Sequence
  FAM labeled probe
cacgccatgcgctgcgg

SEQ ID NO: 4 - DNA, 19 bp, Artificial Sequecne
  primer
tgcggaaccggtgagtaca

SEQ ID NO: 5 - DNA, 20 bp, Artificial Sequence
  primer
cgggtttatccaagaaagga

SEQ ID NO: 6 - DNA, 21 bp, Artificial Sequence
  probe
cggaattgccaggacgaccgg